(19) United States
(12) Patent Application Publication
(10) Pub. No.: US 2026/0216509 A1
(43) Pub. Date: Jul. 30, 2026

(54) NON-INVASIVE NEURO STIMULATION SYSTEM AND METHODS

(71) Applicant: HTK Enterprises, Inc., Plano, TX (US)

(72) Inventors: Janice Ling Walker, Lucas, TX (US); Annette Eileen Murray, Mill Hall, PA (US); Gary L. Byars, Richardson, TX (US); Charles H. Payne, Frisco, TX (US); John W. Muncaster, Dallas, TX (US)

(21) Appl. No.: 19/457,869

(22) Filed: Jan. 23, 2026

Related U.S. Application Data
(60) Provisional application No. 63/750,367, filed on Jan. 28, 2025.

(51) Int. Cl. A61N 1/36

(52) U.S. Cl. CPC A61N 1/36021; A61N 1/36031; A61N 1/36034

(57) ABSTRACT

Provided herein are devices and methods reducing pain or drug dependence from a disease, condition or following a medical procedure of a patient comprises the step of: providing one or more devices having one or more electrodes connected to one or more electric pulse generators that generates a pulse train and a control for controlling the one or more electric pulse generators; administering electric pulses with the one or more electrodes to one or more locations on skin of the patient at one or more reflexology zones or to one or more reflexology zones, or a reflexology zone or a homeostatic nerve point upstream, downstream, or opposite the site of pain; measuring an impedance at the one or more locations; and administering electric pulses at the one or more locations with a lower impedance.

### CROSS-REFERENCE TO RELATED APPLICATIONS

This application claims priority to U.S. Provisional Application Ser. No. 63/750,367, filed Jan. 28, 2025, the entire contents of which are incorporated herein by reference.

### BACKGROUND

Without limiting the scope of the disclosure, its background is described in connection with neuro-stimulation.

The human body suffers many ailments. A neuro-stimulation device has been developed that has been found effective for treating many ailments, and in particular the pain associated with these ailments. Among the ailments treated are acute injuries, speeding recovery from major orthopedic surgery of joints and osteoarthritis, and the associated edema of these ailments, just to name a few. Early versions of the technology were developed in Russia and included an electronic circuit to provide a relatively high voltage, but short duration, electric pulse train to the skin of the patient. The voltage can be high, for example, with a very fast rise and fall time oscillatory pulse but with only a benign amount of energy transferred to the patient. Many of the parameters of the device can be varied to deliver the pulse train in many ways. Devices of this type can be referred to as electro stim devices and typically include a pulse generator and a control mechanism to control the pulse generator whereby the skin of the patient forms part of an LCR circuit absorbing the energy of the pulse. This type of device enables determination of the skin impedance. One example of this type of electro stim device is the SCENAR (Self Controlled Energo Neuro Adaptive Regulator) device found in Russia.

Despite these advances, a need remains for novel methods of treating conditions with targeted neurostimulation.

### SUMMARY

As embodied and broadly described herein, an aspect of the present disclosure relates to a method of reducing pain or drug dependence from a disease, condition or following a medical procedure of a patient comprises the step of: providing one or more devices having one or more electrodes connected to one or more electric pulse generators that generates a pulse train and a control for controlling the one or more electric pulse generators; administering electric pulses with the one or more electrodes to one or more locations on skin of the patient at one or more reflexology zones or to one or more reflexology zones, or a reflexology zone or a homeostatic nerve point upstream, downstream, or opposite a site of pain; measuring an impedance at the one or more locations; and administering one or more electric pulses at the one or more locations until a point stimulation is reached. In one aspect, the method further comprises administering the one or more electric pulses with the one or more electrodes to one or more locations on skin of the patient adjacent a prior location of stimulation, and repeating until the point stimulation is reached at a new location; or determining if two or more treatment location have equal of near equal readings in point stim values, then treat both regions by administering electric pulses to both areas concurrently or in series. In another aspect, the method uses 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or 50 devices. In another aspect, the electric pulses are provided on one or more subsequent days at the one or more locations with the lower impedance that are adjacent to a source of chronic pain, or the reflexology zone or homeostatic nerve points upstream, downstream, or opposite a site of pain, but not at a site of pain. In another aspect, at least one of: the location, a frequency of the electrical pulses, or an energy of the electrical pulses is revised immediately after stimulation, after treating one mor more adjacent areas after treatment, or daily. In another aspect, te two or more locations for treatment are diagonal, contralateral/opposite side, ipsilateral/same side, or checkerboard; or the electric pulses are selected based on one or more characteristics of the patients, wherein the characteristics of the patient are selected from age, weight, length of drug use, extent of pain, number of drugs used, surface area of skin with the lower impedance, location of skin with the lower impedance, or length of time that the patient has suffered from the disease or condition. In another aspect, the disease or condition is selected from at least one of: musculoskeletal conditions, non-healing conditions, post-surgical conditions, ankylosing spondylitis, chronic pain, back pain, sciatica pain, Ehlers Danlos syndrome, fibromyalgia, rheumatoid arthritis, Bell's Palsy, severe headaches, migraines, neuropathic pain conditions, post-traumatic stress disorder (PTSD), anxiety, depression, systemic inflammation conditions, femoral nerve damage, chronic nerve inflammation of sacrum, chronic nerve inflammation of coccyx, chemotherapy induced neuropathy, phantom limb pain, postural orthostatic tachycardia Syndrome (POTS), complex regional pain syndrome (CRPS)/reflex sympathetic dystrophy (RSD), scoliosis, spondylosis, drug dependency, addiction to pain medication, drug addiction, opioid addiction, insomnia, hypersensitivity, to reduce medication(s), connective tissue diseases, or neuropathy. In another aspect, the method further comprises providing two or more electrodes or sets of electrodes connected to the device, the control having: a first preset treatment for controlling the electric pulse generator to generate a first set of electric pulses for delivery to the patient through a first set of said electrodes; and a second preset or multiple presets treatment for controlling the electric pulse generator to generate a second set of electric pulses for delivery to the patient through a second set or multiple of said electrodes; and applying the electrodes based on at least one of: locations with the lower impedance that are adjacent to a source of chronic pain, or the reflexology zone or homeostatic nerve points upstream, downstream, or opposite a site of pain, but not at the site of pain. In another aspect, the method further comprises at least one of: directing one or more additional electric pulses to one or more reflexology zones of hands or feet; mapping the location on a face or spine with highest electrical impedance prior to treatment, and applying the electrodes to one or more head locations, one or more spine locations, one or more scalp locations, or one or more areas of skin with the lowest electrical impedance; subjecting a location near a source of pain or to a reflexology zone thereof based on the mapping, to electric pulses stimulating an area in the reflexology zone to modulate a response wherein a modulated response is one or more of: reducing addiction, reducing pain, reducing patient anxiety, reducing stress, reducing anxiety, and wherein emitted electric pulses are focused or unfocused electric pulses; reducing local inflammation by a direct targeting of the one or more electric pulses at or about a site of inflammation; administering the one or more electric pulses to or in an area of the reflexology zone that modulates a response to the pain to suppress urges to take an addictive medication or opioids, or to reduce withdrawal symptoms; directing an impedance sensing pulse to a skin of the patient and a control for controlling the pulse generator, the impedance sensing pulse at a level below the level necessary for treatment, the impedance sensing pulse applied to the skin at selected locations to measure the impedance of the body to find active areas; placing the electrodes on one or more locations with pain or stiffness and at least one of: moving dynamically the electrodes with exercise, or increasing an intensity of stimulation over a location with a level of pain to lower the pain or stiffness; or moving the one or more electrodes while applying the one or more electric pulses from an initial location of treatment to a position that is at least one of: superior, anterior, posterior, lateral, or inferior from an initial location of treatment. In another aspect, emitted electric pulses are spherical, radial, convergent, divergent, planar, near planar, focused or unfocused and the electric pulses are administered invasively or noninvasively. In another aspect, one or more repeated treatments occur on a schedule over a period of 3, 4, 5, 6, 7, 10 days, or two or more weeks, and treatments can be repeated over time for drug use reduction, pain prevention, reduction of symptoms, anxiety, insomnia, over longer durations of time between repeated treatments. In another aspect, a control that is connected to and controlling an interface allows an operator of the device to select a preset treatment parameter from a menu or menus. In another aspect, the one or more devices have two or more outputs that each connected to one or more electrodes or sets of electrodes that might be stationary or dynamic; or the device further comprises a phase detection circuit to measure one or more components of impedance to isolate a capacitance and resistance, and one or more head locations and one or more spine locations that are selected based on the measured impedance. In another aspect, a location for treatment is first pulsed at a frequency of 2 to 480 Hz on a first day and delivering at least one preset treatment to the patient, wherein a treatment comprises applying electric pulses to one or more head locations and one or more spine locations at between 2 to 480 Hz on one or more subsequent days, until there is a reduction in a disease, condition, pain, or use of drugs. In another aspect, one or more treatments reduce an amount of a prescription drug or drugs used by the patient is reduced by at least 25, 30, 40, 50, 60, 70, 75, 80, 90, 95%, or 100%. In another aspect, a prescription drug or drugs are pain medication(s).

As embodied and broadly described herein, an aspect of the present disclosure relates to a method of reducing pain or drug dependence from a disease, condition or following a medical procedure of a patient comprises the step of: providing one or more devices having one or more electrodes connected to one or more electric pulse generators that generates a pulse train and a control for controlling the one or more electric pulse generators; administering electric pulses with the one or more electrodes to one or more locations on skin of the patient at one or more reflexology zones or to one or more reflexology zones, or a reflexology zone or a homeostatic nerve point upstream, downstream, or opposite a site of pain; measuring an impedance at the one or more locations; and administering electric pulses at the one or more locations with a lower impedance, wherein the disease or condition is selected from at least one of: musculoskeletal conditions, non-healing conditions, post-surgical conditions, ankylosing spondylitis, back pain, sciatica pain, Ehlers Danlos syndrome, fibromyalgia, rheumatoid arthritis, Bell's Palsy, severe headaches, migraines, neuropathic pain conditions, post-traumatic stress disorder (PTSD), anxiety, depression, systemic inflammation conditions, femoral nerve damage, chronic nerve inflammation of sacrum, chronic nerve inflammation of coccyx, chemotherapy induced neuropathy, phantom limb pain, postural orthostatic tachycardia Syndrome (POTS), complex regional pain syndrome (CRPS)/reflex sympathetic dystrophy (RSD), scoliosis, spondylosis, drug dependency, addiction to pain medication, drug addiction, opioid addiction, insomnia, hypersensitivity, to reduce medication(s), connective tissue diseases, or neuropathy. In one aspect, the method further comprises administering the one or more electric pulses with the one or more electrodes to one or more locations on skin of the patient adjacent a prior location of stimulation, and repeating until the point stimulation is reached at a new location; or determining if two or more treatment location have equal of near equal readings in point stim values, then treat both regions by administering electric pulses to both areas concurrently or in series. In another aspect, the method uses 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or 50 devices. In another aspect, the electric pulses are provided on one or more subsequent days at the one or more locations with the lower impedance that are adjacent to a source of chronic pain, or the reflexology zone or homeostatic nerve points upstream, downstream, or opposite a site of pain, but not at a site of pain. In another aspect, at least one of: the location, a frequency of the electrical pulses, or an energy of the electrical pulses is revised immediately after stimulation, after treating one mor more adjacent areas after treatment, or daily. In another aspect, te two or more locations for treatment are diagonal, contralateral/opposite side, ipsilateral/same side, or checkerboard; or the electric pulses are selected based on one or more characteristics of the patients, wherein the characteristics of the patient are selected from age, weight, length of drug use, extent of pain, number of drugs used, surface area of skin with the lower impedance, location of skin with the lower impedance, or length of time that the patient has suffered from the disease or condition. In another aspect, the disease or condition is selected from at least one of: musculoskeletal conditions, non-healing conditions, post-surgical conditions, ankylosing spondylitis, chronic pain, back pain, sciatica pain, Ehlers Danlos syndrome, fibromyalgia, rheumatoid arthritis, Bell's Palsy, severe headaches, migraines, neuropathic pain conditions, post-traumatic stress disorder (PTSD), anxiety, depression, systemic inflammation conditions, femoral nerve damage, chronic nerve inflammation of sacrum, chronic nerve inflammation of coccyx, chemotherapy induced neuropathy, phantom limb pain, postural orthostatic tachycardia Syndrome (POTS), complex regional pain syndrome (CRPS)/reflex sympathetic dystrophy (RSD), scoliosis, spondylosis, drug dependency, addiction to pain medication, drug addiction, opioid addiction, insomnia, hypersensitivity, to reduce medication(s), connective tissue diseases, or neuropathy. In another aspect, the method further comprises providing two or more electrodes or sets of electrodes connected to the device, the control having: a first preset treatment for controlling the electric pulse generator to generate a first set of electric pulses for delivery to the patient through a first set of said electrodes; and a second preset or multiple presets treatment for controlling the electric pulse generator to generate a second set of electric pulses for delivery to the patient through a second set or multiple of said electrodes; and applying the electrodes based on at least one of: locations with the lower impedance that are adjacent to a source of chronic pain, or the reflexology zone or homeostatic nerve points upstream, downstream, or opposite a site of pain, but not at the site of pain. In another aspect, the method further comprises at least one of: directing one or more additional electric pulses to one or more reflexology zones of hands or feet; mapping the location on a face or spine with highest electrical impedance prior to treatment, and applying the electrodes to one or more head locations, one or more spine locations, one or more scalp locations, or one or more areas of skin with the lowest electrical impedance; subjecting a location near a source of pain or to a reflexology zone thereof based on the mapping, to electric pulses stimulating an area in the reflexology zone to modulate a response wherein a modulated response is one or more of: reducing addiction, reducing pain, reducing patient anxiety, reducing stress, reducing anxiety, and wherein emitted electric pulses are focused or unfocused electric pulses; reducing local inflammation by a direct targeting of the one or more electric pulses at or about a site of inflammation; administering the one or more electric pulses to or in an area of the reflexology zone that modulates a response to the pain to suppress urges to take an addictive medication or opioids, or to reduce withdrawal symptoms; directing an impedance sensing pulse to a skin of the patient and a control for controlling the pulse generator, the impedance sensing pulse at a level below the level necessary for treatment, the impedance sensing pulse applied to the skin at selected locations to measure the impedance of the body to find active areas; placing the electrodes on one or more locations with pain or stiffness and at least one of: moving dynamically the electrodes with exercise, or increasing an intensity of stimulation over a location with a level of pain to lower the pain or stiffness; or moving the one or more electrodes while applying the one or more electric pulses from an initial location of treatment to a position that is at least one of:
superior, anterior, posterior, lateral, or inferior from an initial location of treatment. In another aspect, emitted electric pulses are spherical, radial, convergent, divergent, planar, near planar, focused or unfocused and the electric pulses are administered invasively or noninvasively. In another aspect, one or more repeated treatments occur on a schedule over a period of 3, 4, 5, 6, 7, 10 days, or two or more weeks, and treatments can be repeated over time for drug use reduction, pain prevention, reduction of symptoms, anxiety, insomnia, over longer durations of time between repeated treatments. In another aspect, a control that is connected to and controlling an interface allows an operator of the device to select a preset treatment parameter from a menu or menus. In another aspect, the one or more devices have two or more outputs that each connected to one or more electrodes or sets of electrodes that might be stationary or dynamic; or the device further comprises a phase detection circuit to measure one or more components of impedance to isolate a capacitance and resistance, and one or more head locations and one or more spine locations that are selected based on the measured impedance. In another aspect, a location for treatment is first pulsed at a frequency of 2 to 480 Hz on a first day and delivering at least one preset treatment to the patient, wherein a treatment comprises applying electric pulses to one or more head locations and one or more spine locations at between 2 to 480 Hz on one or more subsequent days, until there is a reduction in a disease, condition, pain, or use of drugs. In another aspect, one or more treatments reduce an amount of a prescription drug or drugs used by the patient is reduced by at least 25, 30, 40, 50, 60, 70, 75, 80, 90, 95%, or 100%. In another aspect, a prescription drug or drugs are pain medication(s).

As embodied and broadly described herein, an aspect of the present disclosure relates to a non-transitory computer readable medium for use with a device in a method of reducing pain or drug dependence from a disease, condition or following a medical procedure of a patient comprises the step of: providing one or more devices having one or more electrodes connected to one or more electric pulse generators that generates a pulse train and a control for controlling the one or more electric pulse generators; administering electric pulses with the one or more electrodes to one or more locations on skin of the patient at one or more reflexology zones or to one or more reflexology zones, or a reflexology zone or a homeostatic nerve point upstream, downstream, or opposite a site of pain; measuring an impedance at the one or more locations; and administering electric pulses at the one or more locations until a point stimulation is reached. In one aspect, the method further comprises administering the one or more electric pulses with the one or more electrodes to one or more locations on skin of the patient adjacent a prior location of stimulation, and repeating until the point stimulation is reached at a new location; or determining if two or more treatment location have equal of near equal readings in point stim values, then treat both regions by administering electric pulses to both areas concurrently or in series. In another aspect, the method uses 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or 50 devices. In another aspect, the electric pulses are provided on one or more subsequent days at the one or more locations with the lower impedance that are adjacent to a source of chronic pain, or the reflexology zone or homeostatic nerve points upstream, downstream, or opposite a site of pain, but not at a site of pain. In another aspect, at least one of: the location, a frequency of the electrical pulses, or an energy of the electrical pulses is revised immediately after stimulation, after treating one mor more adjacent areas after treatment, or daily. In another aspect, te two or more locations for treatment are diagonal, contralateral/opposite side, ipsilateral/same side, or checkerboard; or the electric pulses are selected based on one or more characteristics of the patients, wherein the characteristics of the patient are selected from age, weight, length of drug use, extent of pain, number of drugs used, surface area of skin with the lower impedance, location of skin with the lower impedance, or length of time that the patient has suffered from the disease or condition. In another aspect, the disease or condition is selected from at least one of: musculoskeletal conditions, non-healing conditions, post-surgical conditions, ankylosing spondylitis, chronic pain, back pain, sciatica pain, Ehlers Danlos syndrome, fibromyalgia, rheumatoid arthritis, Bell's Palsy, severe headaches, migraines, neuropathic pain conditions, post-traumatic stress disorder (PTSD), anxiety, depression, systemic inflammation conditions, femoral nerve damage, chronic nerve inflammation of sacrum, chronic nerve inflammation of coccyx, chemotherapy induced neuropathy, phantom limb pain, postural orthostatic tachycardia Syndrome (POTS), complex regional pain syndrome (CRPS)/reflex sympathetic dystrophy (RSD), scoliosis, spondylosis, drug dependency, addiction to pain medication, drug addiction, opioid addiction, insomnia, hypersensitivity, to reduce medication(s), connective tissue diseases, or neuropathy. In another aspect, the method further comprises providing two or more electrodes or sets of electrodes connected to the device, the control having: a first preset treatment for controlling the electric pulse generator to generate a first set of electric pulses for delivery to the patient through a first set of said electrodes; and a second preset or multiple presets treatment for controlling the electric pulse generator to generate a second set of electric pulses for delivery to the patient through a second set or multiple of said electrodes; and applying the electrodes based on at least one of: locations with the lower impedance that are adjacent to a source of chronic pain, or the reflexology zone or homeostatic nerve points upstream, downstream, or opposite a site of pain, but not at the site of pain. In another aspect, the method further comprises at least one of: directing one or more additional electric pulses to one or more reflexology zones of hands or feet; mapping the location on a face or spine with highest electrical impedance prior to treatment, and applying the electrodes to one or more head locations, one or more spine locations, one or more scalp locations, or one or more areas of skin with the lowest electrical impedance; subjecting a location near a source of pain or to a reflexology zone thereof based on the mapping, to electric pulses stimulating an area in the reflexology zone to modulate a response wherein a modulated response is one or more of: reducing addiction, reducing pain, reducing patient anxiety, reducing stress, reducing anxiety, and wherein emitted electric pulses are focused or unfocused electric pulses; reducing local inflammation by a direct targeting of the one or more electric pulses at or about a site of inflammation; administering the one or more electric pulses to or in an area of the reflexology zone that modulates a response to the pain to suppress urges to take an addictive medication or opioids, or to reduce withdrawal symptoms; directing an impedance sensing pulse to a skin of the patient and a control for controlling the pulse generator, the impedance sensing pulse at a level below the level necessary for treatment, the impedance sensing pulse applied to the skin at selected locations to measure the impedance of the body to find active areas; placing the electrodes on one or more locations with pain or stiffness and at least one of: moving dynamically the electrodes with exercise, or increasing an intensity of stimulation over a location with a level of pain to lower the pain or stiffness; or moving the one or more electrodes while applying the one or more electric pulses from an initial location of treatment to a position that is at least one of: superior, anterior, posterior, lateral, or inferior from an initial location of treatment. In another aspect, emitted electric pulses are spherical, radial, convergent, divergent, planar, near planar, focused or unfocused and the electric pulses are administered invasively or noninvasively. In another aspect, one or more repeated treatments occur on a schedule over a period of 3, 4, 5, 6, 7, 10 days, or two or more weeks, and treatments can be repeated over time for drug use reduction, pain prevention, reduction of symptoms, anxiety, insomnia, over longer durations of time between repeated treatments. In another aspect, a control that is connected to and controlling an interface allows an operator of the device to select a preset treatment parameter from a menu or menus. In another aspect, the one or more devices have two or more outputs that each connected to one or more electrodes or sets of electrodes that might be stationary or dynamic; or the device further comprises a phase detection circuit to measure one or more components of impedance to isolate a capacitance and resistance, and one or more head locations and one or more spine locations that are selected based on the measured impedance. In another aspect, a location for treatment is first pulsed at a frequency of 2 to 480 Hz on a first day and delivering at least one preset treatment to the patient, wherein a treatment comprises applying electric pulses to one or more head locations and one or more spine locations at between 2 to 480 Hz on one or more subsequent days, until there is a reduction in a disease, condition, pain, or use of drugs. In another aspect, one or more treatments reduce an amount of a prescription drug or drugs used by the patient is reduced by at least 25, 30, 40, 50, 60, 70, 75, 80, 90, 95%, or 100%. In another aspect, a prescription drug or drugs are pain medication(s).

As embodied and broadly described herein, an aspect of the present disclosure relates to a computer-readable storage medium comprising instructions stored thereon that, in response to execution by a processor, causes an apparatus to deliver one or more pulses in a method of reducing pain or drug dependence from a disease, condition or following a medical procedure of a patient comprises the step of: providing one or more devices having one or more electrodes connected to one or more electric pulse generators that generates a pulse train and a control for controlling the one or more electric pulse generators; administering electric pulses with the one or more electrodes to one or more locations on skin of the patient at one or more reflexology zones or to one or more reflexology zones, or a reflexology zone or a homeostatic nerve point upstream, downstream, or opposite a site of pain; measuring an impedance at the one or more locations; and administering electric pulses at the one or more locations until a point stimulation is reached. In one aspect, the method further comprises administering the one or more electric pulses with the one or more electrodes to one or more locations on skin of the patient adjacent a prior location of stimulation, and repeating until the point stimulation is reached at a new location; or determining if two or more treatment location have equal of near equal readings in point stim values, then treat both regions by administering electric pulses to both areas concurrently or in series. In another aspect, the method uses 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or 50 devices. In another aspect, the electric pulses are provided on one or more subsequent days at the one or more locations with the lower impedance that are adjacent to a source of chronic pain, or the reflexology zone or homeostatic nerve points upstream, downstream, or opposite a site of pain, but not at a site of pain. In another aspect, at least one of: the location, a frequency of the electrical pulses, or an energy of the electrical pulses is revised immediately after stimulation, after treating one mor more adjacent areas after treatment, or daily. In another aspect, te two or more locations for treatment are diagonal, contralateral/opposite side, ipsilateral/same side, or checkerboard; or the electric pulses are selected based on one or more characteristics of the patients, wherein the characteristics of the patient are selected from age, weight, length of drug use, extent of pain, number of drugs used, surface area of skin with the lower impedance, location of skin with the lower impedance, or length of time that the patient has suffered from the disease or condition. In another aspect, the disease or condition is selected from at least one of: musculoskeletal conditions, non-healing conditions, post-surgical conditions, ankylosing spondylitis, chronic pain, back pain, sciatica pain, Ehlers Danlos syndrome, fibromyalgia, rheumatoid arthritis, Bell's Palsy, severe headaches, migraines, neuropathic pain conditions, post-traumatic stress disorder (PTSD), anxiety, depression, systemic inflammation conditions, femoral nerve damage, chronic nerve inflammation of sacrum, chronic nerve inflammation of coccyx, chemotherapy induced neuropathy, phantom limb pain, postural orthostatic tachycardia Syndrome (POTS), complex regional pain syndrome (CRPS)/reflex sympathetic dystrophy (RSD), scoliosis, spondylosis, drug dependency, addiction to pain medication, drug addiction, opioid addiction, insomnia, hypersensitivity, to reduce medication(s), connective tissue diseases, or neuropathy. In another aspect, the method further comprises providing two or more electrodes or sets of electrodes connected to the device, the control having: a first preset treatment for controlling the electric pulse generator to generate a first set of electric pulses for delivery to the patient through a first set of said electrodes; and a second preset or multiple presets treatment for controlling the electric pulse generator to generate a second set of electric pulses for delivery to the patient through a second set or multiple of said electrodes; and applying the electrodes based on at least one of: locations with the lower impedance that are adjacent to a source of chronic pain, or the reflexology zone or homeostatic nerve points upstream, downstream, or opposite a site of pain, but not at the site of pain. In another aspect, the method further comprises at least one of: directing one or more additional electric pulses to one or more reflexology zones of hands or feet; mapping the location on a face or spine with highest electrical impedance prior to treatment, and applying the electrodes to one or more head locations, one or more spine locations, one or more scalp locations, or one or more areas of skin with the lowest electrical impedance; subjecting a location near a source of pain or to a reflexology zone thereof based on the mapping, to electric pulses stimulating an area in the reflexology zone to modulate a response wherein a modulated response is one or more of: reducing addiction, reducing pain, reducing patient anxiety, reducing stress, reducing anxiety, and wherein emitted electric pulses are focused or unfocused electric pulses; reducing local inflammation by a direct targeting of the one or more electric pulses at or about a site of inflammation; administering the one or more electric pulses to or in an area of the reflexology zone that modulates a response to the pain to suppress urges to take an addictive medication or opioids, or to reduce withdrawal symptoms; directing an impedance sensing pulse to a skin of the patient and a control for controlling the pulse generator, the impedance sensing pulse at a level below the level necessary for treatment, the impedance sensing pulse applied to the skin at selected locations to measure the impedance of the body to find active areas; placing the electrodes on one or more locations with pain or stiffness and at least one of: moving dynamically the electrodes with exercise, or increasing an intensity of stimulation over a location with a level of pain to lower the pain or stiffness; or moving the one or more electrodes while applying the one or more electric pulses from an initial location of treatment to a position that is at least one of:
superior, anterior, posterior, lateral, or inferior from an initial location of treatment. In another aspect, emitted electric pulses are spherical, radial, convergent, divergent, planar, near planar, focused or unfocused and the electric pulses are administered invasively or noninvasively. In another aspect, one or more repeated treatments occur on a schedule over a period of 3, 4, 5, 6, 7, 10 days, or two or more weeks, and treatments can be repeated over time for drug use reduction, pain prevention, reduction of symptoms, anxiety, insomnia, over longer durations of time between repeated treatments. In another aspect, a control that is connected to and controlling an interface allows an operator of the device to select a preset treatment parameter from a menu or menus. In another aspect, the one or more devices have two or more outputs that each connected to one or more electrodes or sets of electrodes that might be stationary or dynamic; or the device further comprises a phase detection circuit to measure one or more components of impedance to isolate a capacitance and resistance, and one or more head locations and one or more spine locations that are selected based on the measured impedance. In another aspect, a location for treatment is first pulsed at a frequency of 2 to 480 Hz on a first day and delivering at least one preset treatment to the patient, wherein a treatment comprises applying electric pulses to one or more head locations and one or more spine locations at between 2 to 480 Hz on one or more subsequent days, until there is a reduction in a disease, condition, pain, or use of drugs. In another aspect, one or more treatments reduce an amount of a prescription drug or drugs used by the patient is reduced by at least 25, 30, 40, 50, 60, 70, 75, 80, 90, 95%, or 100%. In another aspect, a prescription drug or drugs are pain medication(s).

### BRIEF DESCRIPTION OF THE DRAWINGS

For a more complete understanding of the features and advantages of the present disclosure, reference is now made to the detailed description of the disclosure along with the accompanying figures and in which:

FIG. 1 shows the locations for mapping and/or treating the spine.

FIG. 2 shows the locations for mapping and/or treating the face.

FIG. 3 shows the locations for mapping and/or treating the scalp.

FIG. 4 shows the locations for mapping and/or treating the back of the head.

FIG. 5 show a diagonal relationships treatment map.

FIG. 6 show a Contralateral/Opposite relationships treatment map.

FIG. 7 show an Ipsilateral/Same side relationships treatment map.

FIG. 8 shows the locations for mapping and/or treating the neck.

FIG. 9 shows the locations for mapping and/or treating the abdomen.

FIG. 10 shows the locations for mapping and/or treating the spine at the neck, T7 and sacrum.

FIG. 11 shows a scan of the device over the tops and bottoms of the feet in columns.

FIG. 12 shows representative active sites on the dorsal and volar surfaces of the hands and columns along which the treatment is delivered.

FIG. 13 shows how to find and scan active sites treat active sites which will be sensitive, sticky areas.

FIG. 14 includes additional examples of treatments of the present invention.

FIG. 15 shows examples of treatments of the present invention using a variety of arrays, multiple devices, comb electrode and a soft tissue electrode.

FIG. 16-1 to 16-3 includes additional examples of treatments of the present invention for opioid dependence and other dependencies.

FIGS. 17A and 17B shows additional dynamic and static stimulation points used with the apparatus and methods of the present disclosure. FIG. 17A the frontal dynamic and static stimulation points and FIG. 17B the distal dynamic and static stimulation points.

### DETAILED DESCRIPTION OF THE INVENTION

While the making and using of various aspects of the present disclosure are discussed in detail below, it should be appreciated that the present disclosure provides many applicable inventive concepts that can be embodied in a wide variety of specific contexts. The specific aspects discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims.

As used herein, the term “Point-stimulation value” or “Point-stim value” refers to a signal to the operator that a certain level of impedance change has been measure by the InterX device. Examples of devices for use with the present disclosure include those taught in U.S. Pat. Nos. 8,948,879 and 9,630,003, assigned to the present Applicant, and incorporated herein by reference in its entirety as regards the type of device used herein.

As used herein, the term “reflexology zones” refers to a system for formulating relationships between various parts of the body. Specific locations related to a broader area in the body.

As used herein, the term “homeostatic nerve point” refers to a series of predetermined points relating to major nerve pathways to provide a logical and systematic approach for establishing an adjunctive InterX device treatment to optimize patient response, symptomatic relief and functional outcomes. There are 23 bilateral points and one point on the spine at T7, a total of 47 points which can effectively be treated with the InterX device.

As used herein, the term “activity reading” or “AR” refers to an objective measure of skin impedance that is used to provide relative measures between different points on the skin.

As used herein, the term “Organ channels/Source points” refers to the point areas around the wrists, feet and ankles that correspond to a manage organ meridian.

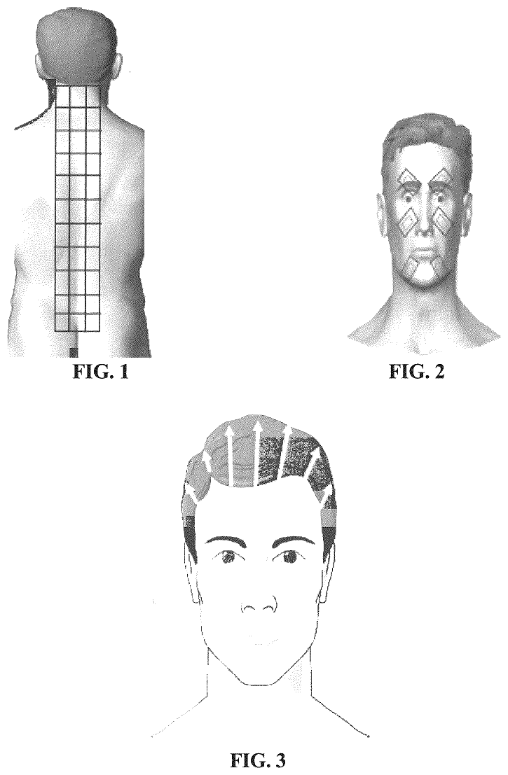

Spine Mapping and why: Spine Assessment Protocol: What is the Spine Assessment Protocol. This method identifies and treats the levels of the spine that are presenting with the greatest sympathetic skin response. As shown in FIG. 1, the area scanned is a column reaching from the hairline to the sacrum with a width of approximately three InterX (InterX, USA, www.interx. com) electrodes (center, left and right paraspinals). The protocol also includes six trigeminal points on the face.

The Spine Assessment protocol should be used at the beginning of a treatment course. Mapping the spine will localize symptoms and will reveal the immediate areas to treat via the highest point stim numbers. It is good to perform a spine assessment at least every 10 visits or if a patient flares and presents with dispersed pain throughout their body. It will calm down their symptoms and reorganize the CNS.

Typical Presets. Patients on multiple medications, poor sleep, elderly or deconditioned, and hypersensitive should be mapped at a first frequency, e.g., 5, 10, 15, 20, 25, 30, 35, 40, 45, 50, 55, or 60 Hz setting. Patients that are not on medications, are fairly active and in good condition, sleep 6+ hours a night can be mapped at a higher frequency, e.g., at a 30, 35, 40, 45, 50, 55, 60, or higher setting. If the user has been treating the patient for multiple visits and are doing a reassessment it is possible to use 180 Hz unless they are very flared, and the user wants to calm the system down then use 15 to 60 Hz.

As shown in FIG. 1, the spinal treatment area is separated into adjoining rows of three electrodes width beginning on the neck at the hairline. The aim is to scan each of the three electrode positions in a row (center, left, right) using the activity reading (AR) and then to target the most active (highest AR) with a Point-Stim (PS). The Point stim value and location of that position should then be recorded. Apply the same scan and target to the next row (below the previous) and scan each of those electrode positions, targeting the most active. Continue down the spine in the same way, recording the point stim value and position for each active site in a row until the whole spine has been completed. To finish the spinal part of the treatment, target the three most active sites on the spine for one minute each. The three most active sites are where the three highest Point-stim values were recorded during the initial Scan and Target. One to 2 sites should decrease in value and one or two sites will remain high. If the user has multiple devices, it is possible to pad the highest areas with the flex arrays while mapping the face. Pad is set on 15-60 Hz.

Switching back to 15 to 60 Hz preset scan each point on the face a point stimulation is reached, record the number. Repeat for the middle row of points and the upper row of points. Then target the most active facial point for 2 minutes dragging the device toward the ear, thereby treating that specific dermatome region of the face. If 2 areas have high values, then treat two regions for two minutes dragging the device toward the ear.

Preparation. As shown in FIG. 1, mark out the level of each row on the spine using a pen suitable for skin application. In general, 3 finger width equals the width of the device to mark the spine from neck to sacrum. There is no need to mark horizontally.

Turn the device on, and select the chronic icon on the screen and choose 15 or 60 pulses per second (pps) preset.

Spray the skin with a light mist of water, apply the device to the center mark of the neck. Increase the intensity to a comfortable but moderate tingling. Use the + button to increase intensity. Do not change the intensity once it is set for this treatment unless it gets too strong at any location. Record what the setting of the preset and the intensity is. If the intensity needs to be reduced at any one level because it is too strong, the user will want to go back to the original setting at the next level if it is not too strong. The goal is to see where in the spine the point stimulation comes out the highest at the same setting throughout the mapping process.

Spine: Scan and Target. Take an Activity Reading (AR) at each point on the first row below the hairline. Apply a Point-stimulator (PS), device will beep if the sound is on at the position that delivered the highest AR. Record the Point-stim value. It may take a full 60 seconds for a point stim on chronic patients. Now take ARs for the second row and treat the highest one with a point-stim. Repeat this process for every row down to the sacrum. Record each level.

Spine: Secondary Target; Three most active points. Check the documentation and identify the three points which delivered the highest point-stim values. Target each of these for one minute. Then treat the highest one of those three in a grid like fashion. Often the highest point is at the cervical spine regardless of where the patient's pain is. If the patient has moderate GI issues the number will be highest in the lower to mid thoracic region. The user can pad with the flex array on the two highest areas, or if the user has multiple devices while the user scans the face using a 15-60 Hz setting for the pads.

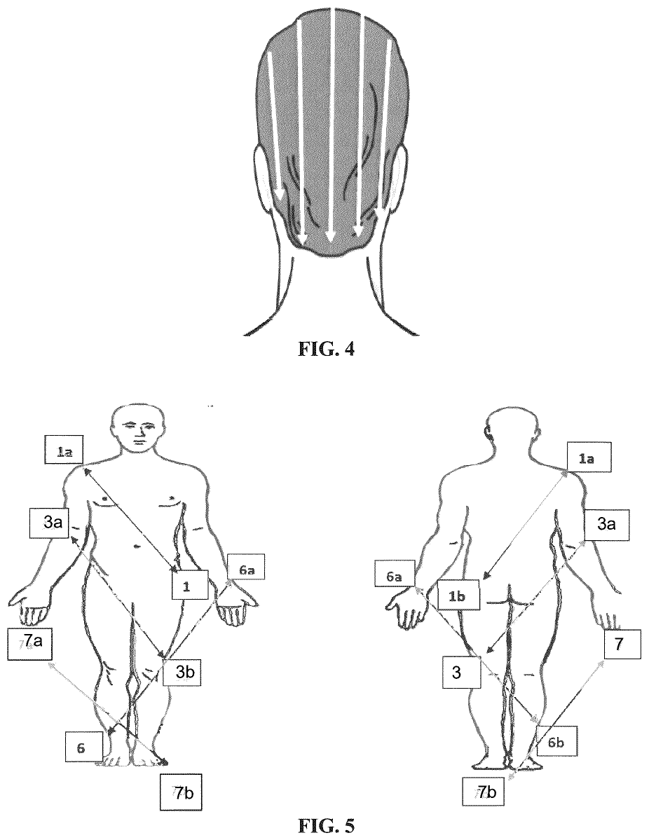

Treatment of the Face. FIG. 2 shows the locations for mapping the face. Switching back to 15 to 60 Hz preset scan each point on the face shown in FIG. 2 until a point stimulation is reached, record the number. Repeat for the middle row of points and the upper row of points. Then target the most active facial point for 2 minutes dragging the device toward the ear, thereby treating that specific dermatome region of the face. If 2 areas have high point stim values, then treat two regions for two minutes dragging the device toward the ear.

Face: Scan and Target. Lightly dampen the lower level of the face with water. Turn the device on 15 Hz setting and slowly turn up the intensity with the + button until the patient feels a comfortable tingling. Usually it is only 3-5% reading. Record the setting and the intensity level. Apply a Point-stim at each location on the face dampening each location with water. Record the Point-stim value. Repeat this process for each of the three rows of points keeping the intensity the same if possible. Be aware that the face can be very sensitive at the forehead or cheeks for some people.

Face: Secondary Target; Most active point. Target the most active point for two minutes dragging the device toward the ear in that region. If two areas are near equal in point stim values, then treat both regions.

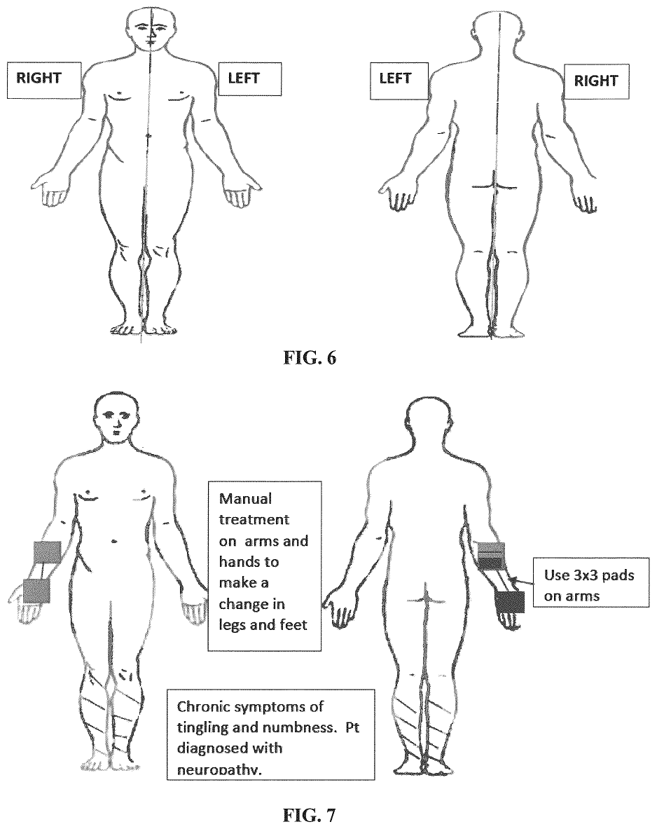

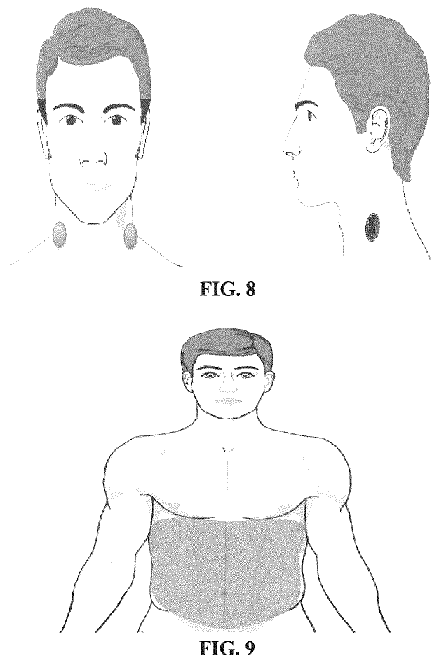

Treatment of the scalp. Use the comb electrode for this application as shown in FIGS. 3 and 4, which show a view of the face and the arrows point to the travel of the electrode(s) along the scalp (FIG. 3), and with the view of the back of the head (FIG. 4) and the line of travel of the electode(s). Place the electrode onto the area around the anterior hairline to set the intensity. This ensures good skin contact for the initial sensation to be correct. Comb the electrode very slowly through the hair two or three times from the anterior hairline to the posterior hairline. The points to target are where the patient feels the stimulation stronger. Target any sensitive points for 1 minute each. The user may need to reduce the intensity while treating these points. Continue like this until the whole scalp has been scanned and active points targeted. Some patients may have delayed sensation so it will be necessary to hold the comb in one spot for 10 seconds before the stimulation penetrates the scalp. This delayed response will shorten with treatment.

Plug in the comb electrode: 1. Select the Chronic preset, 2. Set intensity on the Anterior hairline, 3. Scan slowly through the scalp 2-3 times along each line, 4. Target active areas (sensitive) points for one minute, and/or 5. Look for red areas on the scalp and treat one minute.

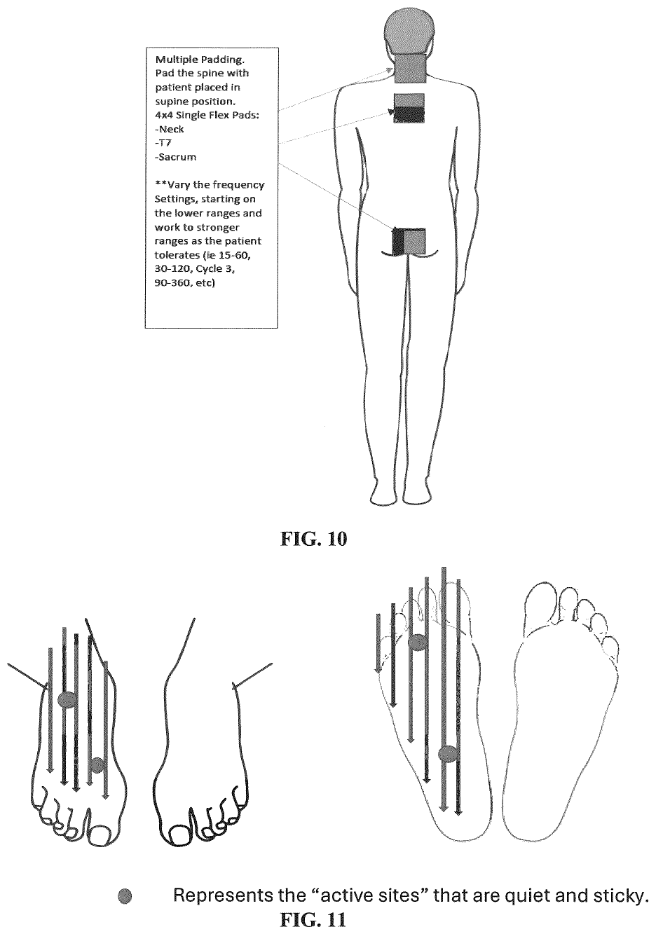

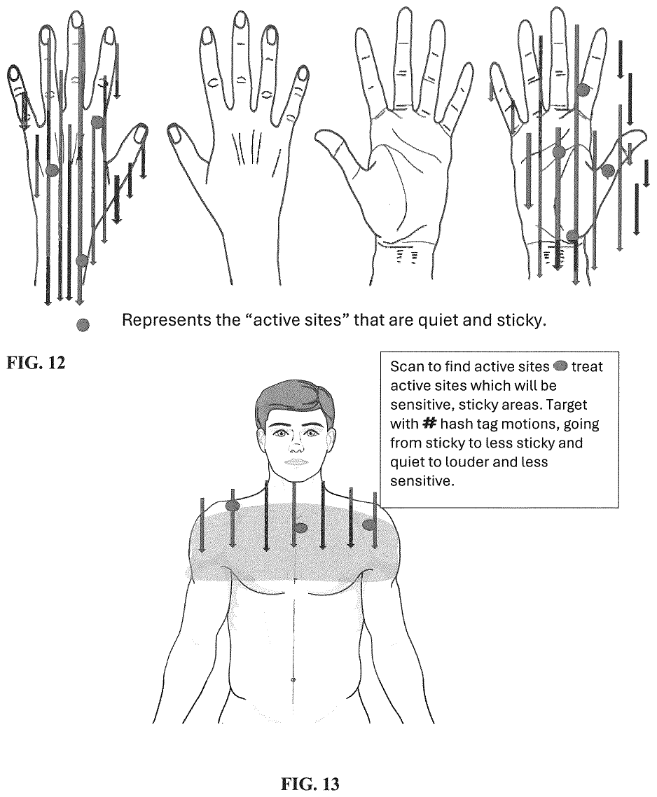

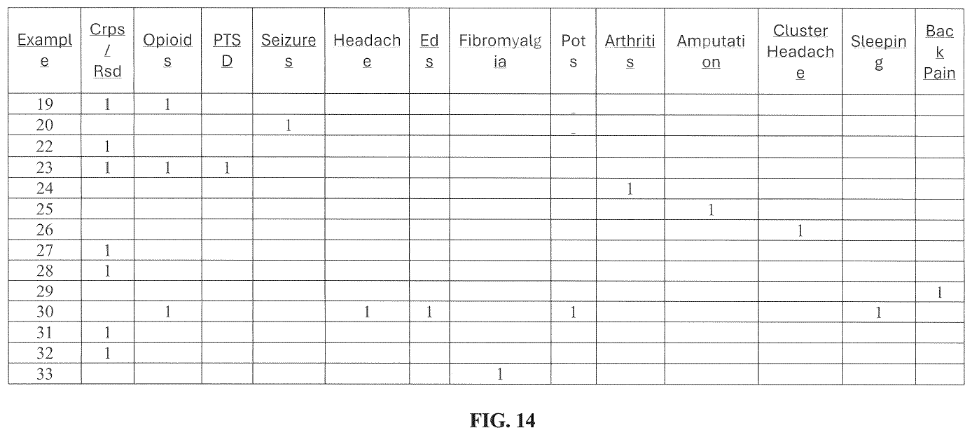

There are many relationships in the body. Treating reflexively is one of the most effective ways to treat with InterX. These reflexive treatments include diagonal relationships (FIG. 5), contralateral/opposite relationships (FIG. 6), ipsilateral/same side relationships (FIG. 7). The strongest relationship to the injured part as it becomes chronic, is most often the diagonal relationship.

Diagonal Relationship. If the injured side has already been treated or the user is not getting the desired result, or the location cannot be reached or is on the injured side (i.e., casted or the patient is too hypersensitive to touch, use the diagonal), as shown in FIG. 5: Right Shoulder (1a) to Left Hip (1b); Left Shoulder (2a)to Right Hip (2b); Right Elbow (3a) to Left Knee (3b); Left Elbow (4a) to Right Knee (4b); Right Wrist (5a) to Left Ankle (5b); Left Wrist (6a) to Right Ankle (6b); Right Hand (7a) to Left Foot (7b); and Left Hand (8a) to Right Foot (8b).

Contralateral (opposite side) treatment. Generally, the second strongest reflexive relationship (i.e., pain in the right shoulder, treat the left shoulder) is shown in FIG. 5. The Right to Left Relationship is the second strongest and very effective. One example of an effective treatment would be to manually treat the contralateral opposite or pad the opposite side to the injury while manually working the diagonal with a second device.

Ipsilateral Treatment-Same Side Treatment. The third strongest reflexive treatment is the Ipsilateral or Same side treatment, as shown in FIGS. 6 and 7. An example of treatment using the Ipsilateral treatment would be if the patient had moderate edema in both feet with a diagnosis of neuropathy. The user can effectively treat both wrists and hands to make a change in swelling and discomfort from the numbness/tingling. A good treatment plan would be to: use flex array pad on arms/hands or manually treat, and/or treat face to incorporate systemic treatment.

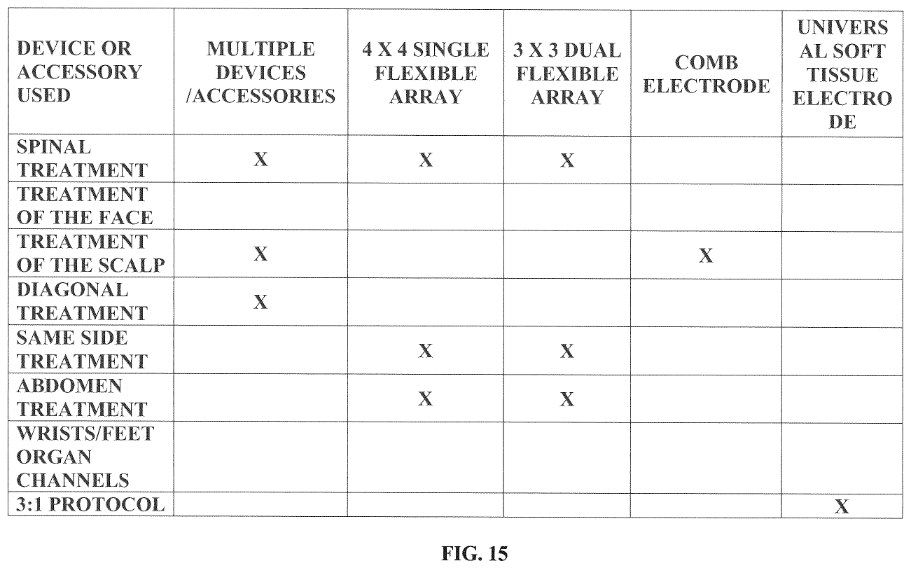

Protocol or treatment (“Little Wings”) at the neck, as shown in FIG. 8. Perform three good contractions on each side of the neck. The method is to balance contractions, at the points designated with an oval in FIG. 7.

1. Choose Preset 3:1, 2. Set Intensity on crest of Trapezius, 3. Slide electrode along the muscle to the treatment point indicated above, 4. Increase intensity to produce a firm muscle contraction, 5. Hold still until three good contractions are complete, and/or 6. Repeat 2-5 on the opposite side. Presets are stimulation patterns that are a range of settings delivering pulses per second as either continuous, burst, variable or cycles.

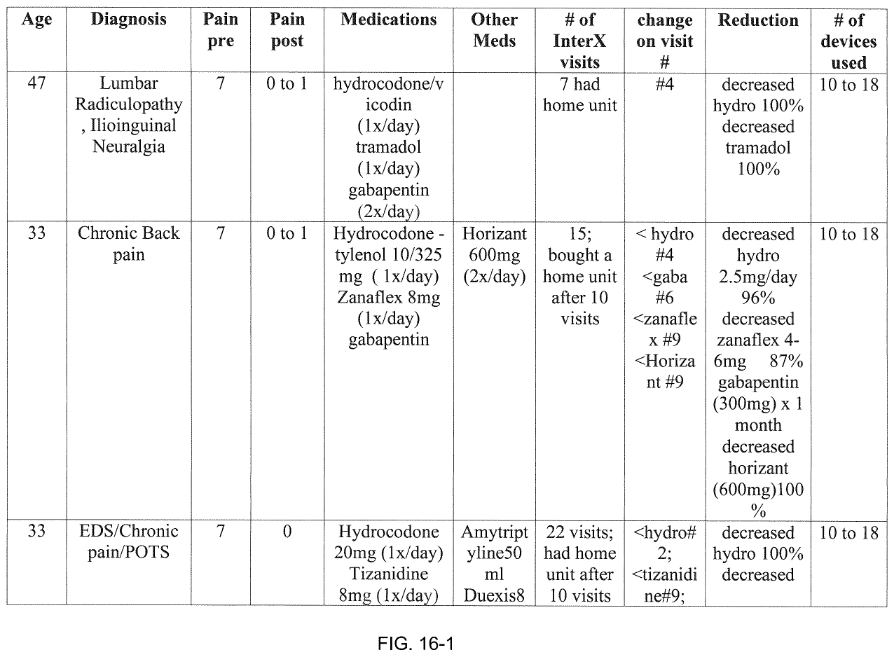

Areas for mapping and treating the abdomen are shown in FIG. 9. Preparation. Turn the device on. Select the chronic icon on the screen and choose the recommended preset. Place the electrode onto the shoulder and increase intensity to a comfortable tingling. Strong, but not uncomfortable. Use the + button to increase intensity.

FIG. 9 shows the scan and target areas for the treatment of the abdomen. Slide the electrode very slowly over the whole treatment area noted above in red. Anywhere that the electrode drags on the skin or the user feels the stimulation stronger, apply a point stimulator. Then rub the electrode back and forth over the point vigorously for 30 seconds. It is possible to find 6 to 10 points to treat in this way.

The user may also scan the abdomen and determine one or two of the most active areas and target the stimulation to these points using the dual or single flexible array. Having scanned the whole area for 10 minutes with the handheld device the user can target the specific points for 10 minutes using the variable preset and the Flexible Array. Settings: 15 Hz to 60 Hz; increase as patient tolerates.

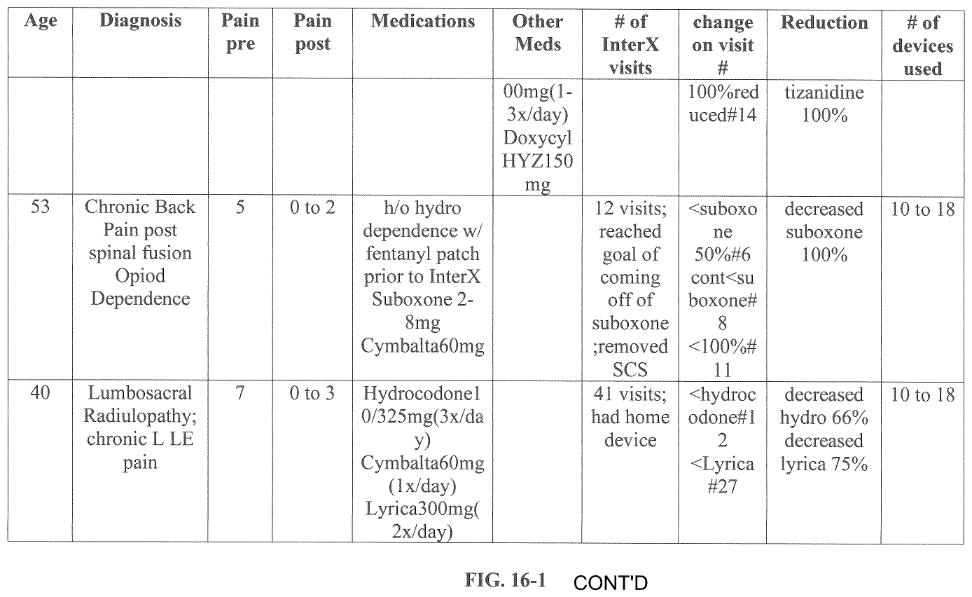

FIG. 10 shows the locations for mapping and/or treating the spine at the neck, T7 and sacrum.

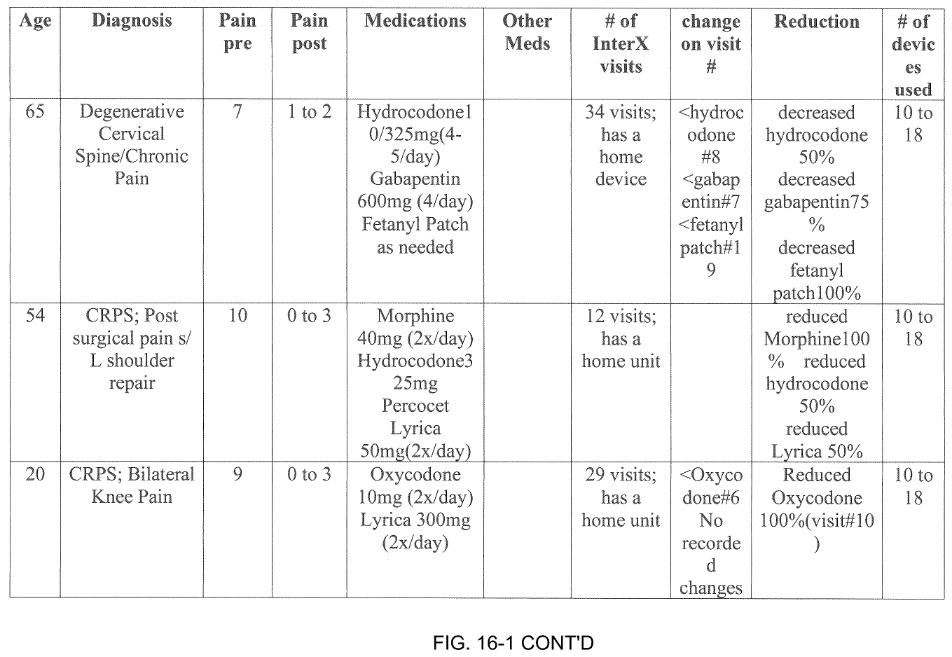

FIG. 11 shows representative active sites on the dorsal and volar surfaces of the feet and columns along which the treatment is delivered. Description of application: use InterX device and scan the device over the tops and bottoms of the feet in columns (FIG. 11, see above; do this left and right) to make sure you cover the whole surface of the dorsal and volar surface of both feet. Stop where the device gets sticky and quiet, on the “active sites”. Hold in these places for 30-60 seconds along the whole surface of foot on the top and bottom, followed by working the area in a “tic-tac-toe” way. The “active site” will lessen in stickiness and will become louder, when this happens, the treatment is done. This treatment is effective for the whole body, internal system/organs, causing whole body relaxation. Other directions are also possible.

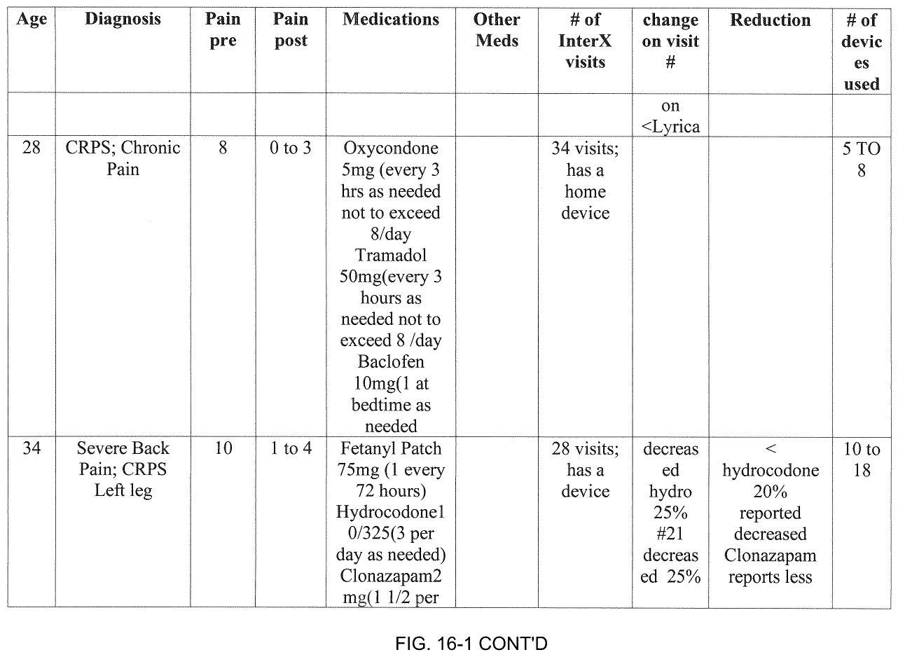

FIG. 12 shows representative active sites on the dorsal and volar surfaces of the hands and columns along which the treatment is delivered. Description of application: use InterX device and scan the device over the tops and bottoms of the hands in columns (see above; do this left and right) to make sure you cover the whole surface of the dorsal and volar surface of both hands. Stop where the device gets sticky and quiet, on the “active sites”. Hold in these places for 30-60 seconds along the whole surface of both hands on the top and bottom, followed by working the area in a “tic-tac-toe” way. The “active site” will lessen in stickiness and will become louder, when this happens, the treatment is done. This treatment is effective for the whole body, internal system/organs, causing whole body relaxation.

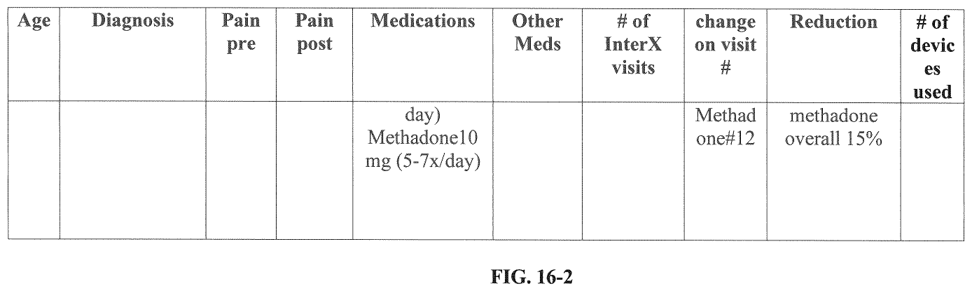

FIG. 13 shows how to find and scan active sites treat active sites which will be sensitive, sticky areas. Target with hash tag motions, going from sticky to less sticky and quiet to louder and less sensitive. Chest Treatment. In addition to treating the abdomen, the chest treatment is very effective in restoring gut health and also can be a reflexive treatment for lower back pain and aides in shoulder pain treatment. This area is usually sensitive when treating so start on the lowest setting 15 Hz. The frequency can be increased on successive treatments as tolerated. The chest area starts from the sternal notch and goes to just above the nipple line. Be sure to cover the entire chest to the tip of the shoulder on both sides. It is easier to do one side at a time. Dampen the skin with water, scan the chest searching for sensitive or sticky areas or the highest reading; these are the active sites. Target these areas for 30-60 seconds followed by dragging the device in a hash-tag pattern until they become less sticky. Do this for the entire chest. Often the skin will get red with treating the active sites. Be careful not to irritate the skin as it is a sensitive area.

Face treatment. The Trigeminal Protocol. This protocol uses a series of predetermined points on the face that relate to both trigeminal nerve outlet points and acupuncture points. There are six points used in this protocol. The six points are the strongest entry points or the face treatment, but the user can also scan the device toward the ear to treat the entire region.

The trigeminal point protocol is very effective in making a parasympathetic shift to the body. The user will notice the patient will get relaxed even sleepy treating the face. It is an effective way to reduce nausea, anxiousness, inability to sleep, and each region of the face will address specific areas of the body.

The trigeminal point protocol should be used during the treatment sessions as an adjunct to treatment on other areas. Doing the protocol first often relaxes the patient. It can be used every treatment session if time allows or is recommended every 2nd or 3rd visit.

Which Points Should Patient Use? Treat all six points on the face. The user may find that one point is more reactive than others either by the fact it feels more sensitive or by using the point stim value. If this is the case, the user may wish to treat on this point for longer than the others. Also, the user will want to treat longer on the corresponding region on the face for the body part that the user wants to affect.

Typical Presets. To begin with use the chronic preset of 15 or 60 but if the patient is hypersensitive the preset 15 pps may be more appropriate. If the protocol is used regularly the user will want to vary the presets by increasing or lowering the frequency or preset with each treatment. Usually, 15 pps will help induce sleep, higher frequencies can be used for acute purposes, such as an acute headache.

Dampen the face. Place the electrode onto the first point at the chin (V3) and increase the intensity to a very comfortable tingling sensation. Treatment on the face should be done fairly gently. Treat each point for two minutes. The user may need to turn the intensity down when moving the devices from one point to the next and then increase it back up again to the correct level for each point. The top two points are just under the eyebrow either side of the bridge of the nose. The user may only be able to contact the skin with half of the electrode. This is generally fine as long as the user reduces the intensity before making contact with each point. After treating the 6 points for 2 minutes, go back to the highest point stim area and scan with the device toward the ear to treat the entire region. The user may also want to treat the region of the face that is related to their problem for an additional 5 minutes. Example, if the patient has foot pain, treat V1 for an additional 5 minutes.

Homeostatic points. Nerve Point Protocol. This protocol uses a series of predetermined points relating to major nerve pathways to provide a systematic approach for establishing homeostasis or balance in the body. They are also known as homeostasis points. There are 23 bilateral points and one point on the spine at T7, a total of 47 points which can effectively be treated with InterX.

The nerve point protocol is an effective way to establish positive outcomes in patients who are becoming chronic or are already chronic in their condition. It is recommended to use this protocol by the third day of acute treatment or the second day of chronic treatment after the spine and face assessment.

Locating the treatment points. Use the chart to identify the location of each point. For points that are a little less specific to locate, move the device slightly until the strongest stimulation is felt in that area or the highest AR number. Sometimes it is necessary to pick the device up and replace it on the spot to get the highest value.

Typical Presets. To begin with use the chronic preset of 60 pps but if the patient is hypersensitive, elderly, or on multiple medications the preset 15 pps may be more appropriate for the first treatment. During successive treatments, you can increase the frequency as the patient tolerates.

Treatment at each point. Having identified the location of point no. 1, spray with water, set intensity to a comfortable tingling sensation directly on the point to be treated. Treat the point to obtain a point stim and record the number. If the point stim occurs quickly (less than 30 seconds) stay on the point for the full 60 seconds. Then remove the device from the treatment point, reduce intensity back down and then repeat the procedure on the same point on the contralateral side. If you have two devices you can treat both sides at the same time which saves significant treatment time and improves outcomes. If time allows treat points 1-10 in one visit.

Organ channels/Source points. There are 6 points around the wrists and feet that are entry points for energy that are specific to organ meridians. These are determined from traditional Chinese medicine TCM. It has been seen clinically that treating these points is very effective in reducing pain. When all four extremities are treated in one session it is often the most effective pain reducing treatment regardless of the patient's condition.

It is suggested to use this protocol on chronic patients. If the patient is elderly or deconditioned, the user should only treat one wrist and one foot in the same treatment. Treating all four body parts in one treatment has a strong detoxing effect for the first time treating the organs. The patient should be informed of the effects, so they are not caught off guard.

Begin the organ treatment on the second or third day of treatment with chronic patients. As treatment sessions continue, use the organ points frequently to reduce strong pain flare ups, GI disturbances, or to help with sleep. It is recommended that the organ points get treated at least in part every 2-3 visits.

Typical Presets. To begin with use the preset 15 pps if elderly or 60 pps. As the patient continues with successive days of treatment, the settings can be increased to higher frequencies as tolerated going from low and gradually increasing up to 240 pps and 360 pps.

Treatment at each point. Locate the point on the wrist or foot according to the chart, spray with water, set the preset on the device to 15 or 60 pps, turn up the intensity with the + button until the patient feels the stimulation. This will vary greatly from the palm side of the wrist to the dorsal side so go slowly on increasing the intensity. The sensation also varies greatly from point to point on the foot. Treat each point for 60 seconds and record the point stim number. If you have two devices, the organ treatment time will be significantly shortened, and the user should be able to do both wrists and feet in one treatment. Again, treating all four parts in one session is very effective in reducing any pain, even disc pain.

Scalp treatment. This protocol is an extension of the trigeminal protocol to further target the cranial nerves. It involves scanning the scalp area and targeting stimulation to active points. If patients are prone to headaches the scalp treatment may often elicit a strong headache with the first treatment. If this occurs, inform the patient that the headache will pass in a day. If the patient did get a headache with the first treatment, then the second treatment usually elicits a milder headache and the third treatment is usually pain free. The result of a headache indicates that the scalp is an effective treatment location for this patient and they usually get good results with treatment.

This protocol should be employed on patients with multi-system or neuropathic dysfunction or patients that are not responding to other treatment. The scalp treatment represents the entire body like the face treatment. The back half represents the spine, the front half the extremities. It is very effective in reducing pain that is difficult to change.

The scalp protocol should be used during the treatment sessions as an adjunct to treatment on other areas. It should be noted that patients can sometimes experience a flare-up so this protocol should only be used after 3 or 4 treatments have been completed.

Treatment at each point. To begin with use the chronic preset of 60 pps. If the patient is hypersensitive or very prone to headaches just do half of the scalp in one session. Once the patient tolerates 60 pps then you can increase the frequency to 180 pps on the next scalp treatment. Once that is tolerated then go to 240 pps.

Typically, use the comb electrode for this application. Place the electrode onto the area around the anterior hairline to set the intensity. This ensures that you have good skin contact for the initial sensation to be correct. Comb the electrode very slowly through the hair two or three times from the anterior hairline to the posterior hairline. The points to target are where the patient feels the stimulation stronger. Target any sensitive points for 1 minute each. You may need to reduce the intensity while treating these points. Continue like this until the whole scalp has been scanned and active points targeted. Some patients may have delayed sensation so it will be necessary to hold the comb in one spot for 10 seconds before the stimulation penetrates the scalp. This delayed response will shorten with treatment.

Abdomen treatment. This involves the treatment of the cutaneous nerves around the abdomen to elicit a centrally mediated response. The nerves on the abdomen wrap around to the thoracic and lumbar spine. It is a very effective treatment for thoracic and low back pain. It also is an indirect treatment to the brain and hypothalamus.

This involves the treatment of the cutaneous nerves around the abdomen to elicit a centrally mediated response. The nerves on the abdomen wrap around to the thoracic and lumbar spine. It is a very effective treatment for thoracic and low back pain. It also is an indirect treatment to the brain and hypothalamus.

Use this protocol as a supplement to treating pain patients who are slow to respond to treatment or who have secondary issues as a result of their pain.

Lightly spray the abdomen with water, scan the treatment area as normal starting at the base of the sternum going left to right and target the stimulation to Active Sites as you find them. As this is a larger surface area there will be more points to treat. Note that patients with stomach issues will be sensitive on the left side of the abdomen and those with liver or gall bladder issues will be sensitive on the right side. Scan below the belly button at least one width of the device to effectively treat for lower abdominal issue or lower back pain.

Typical Presets. To begin with use the chronic preset of 60 pps but if the patient is hypersensitive the preset 15 pps may be more appropriate. As the patients gets less sensitive with successive treatments, you can increase the frequency to 180 and 240 Hz.

What is the 3:1 Protocol? Why do you do 3:1.

This protocol involves the stimulation of the nerves serving the Trapezius and Levator Scapulae muscles to create a muscle contraction.

This protocol should be employed on patients with muscle tension in the neck area. It can also be used for any other patient who would benefit from the relaxation effect of the treatment, especially patients experiencing upper limb pain.

The 3:1 protocol can be used at the beginning or end of a treatment. This protocol should be avoided if there is any shoulder joint instability following injury or surgery.

Typical Presets. Use the 3:1 preset, which you can access by selecting the book icon and scrolling down to the 11th preset in the list. The preset modulates amplitude with it being on for 3 seconds and off for 1 second.

Treatment at each point. Place the soft tissue electrodes onto the upper cervical spine and set intensity to a comfortable tingling. Once the patient can feel the stimulation slide the electrode along the cervical muscle until you reach the point just in front of the Trapezius and just behind the Sternocleidomastoid muscle. With your free hand palpate the occipital area to confirm appropriate contraction of the trapezius.

The correct treatment point for each patient will be different so you may need to move or turn the electrode to get the correct contraction. This should be an elevation and retraction of the scapula. Hold the electrode still with moderate pressure until the desired contractions are complete. You may need to increase or decrease intensity to ensure the correct level of contraction. It should be strong enough to elevate the scapula.

Modifications of the methods.

Mapping the spine: if a patient flares up with pain all over the body or comes in and “just doesn't feel good” or you are not sure what to do, Remap the Spine & Face. Use a lower setting like 60 or 15 Hz and their body should calm down.

Scalp treatment: If a patient has left sided pain, treat the right side of scalp. Same for right sided pain. The scalp treatment really calms neuropathic (nerve) pain down. Work on lower frequency 15 or 60 Hz on sensitive people, elderly or children to begin with. Higher cortex of the brain responds to higher than 180 Hz frequency, so mainly use 180, 240 and 360 Hz.

Face treatment: Shifts the body from a sympathetic (fight or flight) place to a parasympathetic place (place of healing, rest, improves digestion/gut). Start on lower frequency and move up as the patient tolerates (15, 60, 180, 240 Hz, etc.). The face treatment also calms neuropathic (nerve) pain down.

Organ treatment: Treat around the wrists and feet/ankles on organ points when the patient has GUT/GI issues, spine pain, works well on disc pain patients. Start on lower frequencies and work up as the patient tolerates.

Pirigov Ring: Place the device at the back of the neck just below the hairline for 1 minute. Slowly move the device around the neck keeping good contact on the skin; when you get the back of the neck, hold the device for another 1 minute. Do this 4 times. This treatment helps with neck pain and helps to lower inflammation all over the body.

Abdomen treatment: Treat in a downward motion for constipation, and upwards motion for diarrhea, treat horizontally for normal bowel consistency

Severe GUT/GI issues: usually there is bloating, pain and maybe nausea. Treat all 4 extremities on organ points/channels and treat the abdomen as well.

Severe headache: Treat the abdomen and pad the sacrum, treat the face, organ points/channels and treat HSP #12, specifically, 3:1 is a variable preset that has 3 pulse on and 1 off.

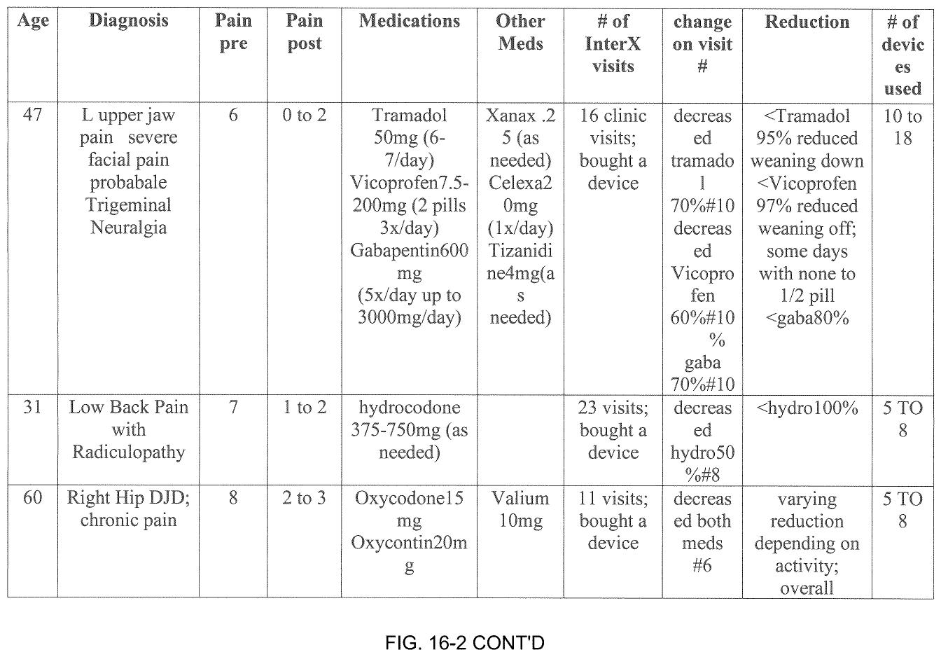

FIG. 14 shows how to find and scan active sites treat active sites of the vagus nerve.

Vagus Nerve Treatment. The vagus nerve originates from cranial nerve #10 and winds from the neck down the side of the torso and supplies all the internal organs with signals from the brain. When this nerve is overactive it can make you have an all body sweat response, blurred vision, fainting, pale color. When it is underactive the internal organs become sluggish, digestion can nearly stop, vocal cords can be affected, breathing can be shallow. Most people with vagus nerve issues are underactive and require stimulation to the vagus nerve to get it working correctly. There are implanted vagus nerve stimulators and external ones. The InterX works very well to stimulate the vagus nerve externally.

Treatment: Lightly dampen the skin, place the dome or electrode head of the device directly on the vagus nerve located on the neck in front of the ear. You will feel a strong vibration when you are on it. Start with setting 15 Hz or 60 Hz and treat for 2-3 minutes. You can treat both sides of the neck but know that the right side also can lower your heart rate as it directly affects the heart more than the left side. It is recommended to not treat more than 5 minutes as to not over stimulate the vagus nerve. Higher settings can also be very effective especially if the subject is feeling sick.

### Example 1

### Chronic Treatment

The following is an example of a protocol or method of the present invention for use with chronic conditions. A chronic condition is a condition that one week old or older.

First series of treatments: Day 1-14.

Day 1: Evaluation(Spine and face mapping eval form & instructions in addendum)

Treat:
Map spine.
Map face use low frequency 15 or 60 Hz on both spine & face. Record the numbers.

If the patient is geriatric and deconditioned, pad only the arms on 15 Hz; map the spine at 15 Hz, Children: use low frequency at 15 Hz, If <70 years old (y.o.) and in fair condition pad arms and legs 15 or 15-60 Hz. See pad chart for locations also pad reflexively. Do not pad directly on the involved body part.

Day 2: Assess tolerance to treatment & symptom changes—sleep, bowel elimination, gut, flares (Herxheimer reaction). If the patient is very tender to touch/hypersensitive, stay at 15 Hz, use less pads, especially on sensitive areas. If no change then increase frequency to 60 Hz.

Treat:
Homeostatic/nerve point treatment. (See addendum). Treat nerve points 1-12both sides.
Organ points, face 15 or 60 Hz. (see addendum for diagrams) ** Record the numbers***
Pad reflexively for involved body part. (see addendum for reflexive diagram).
Pad the spine and extremities 15-60 Hz or if young person can increase frequency setting to Cycl 3 or 30-120 Hz.
Pad the spine where the numbers came out the highest on the spine treatment eval form from Day 1.

Tips: If tolerates well pad above and below involved part but stay off the part directly. If involved part flares with any treatment stay off the part including above and below it. To calm the flared part, treat reflexively & treat the face. Address supplements for patient condition, continue to address supplements with each visit.

Day 3: Address tolerance to treatment and symptom changes:

If flared stay at low frequency, address conditions of flare (i.e., no sleep, overindulgence of trigger food, result of treatment).

Treat:
Directly on body part of flare if it is not the injured part, if the injured part flares then stay off that area. Do not pad above or below it. If it does not flare then pad above and below it. Do not treat involved body part directly until the pain starts to subside.
Continue on Homeostatic/Nerve Points 13-24 (60 Hz).
Organ points (60 Hz).
Face points again; increase frequency as tolerated to 60 Hz or up to 180 Hz.
Padding the body: reflexively, over skin changes, spine, extremities, any new pains on 30-120 or cycle 3.

Tips: When not treating the spine manually then use the pads on areas where numbers come out the highest or according to nerve root and best reflexive areas. (see padding chart for spine).

Day 4: Continue to assess tolerance to InterX, flares, new symptoms or old pain returning (Hering's Law of Healing-see addendum). Continue to increase frequency as tolerate or decrease frequency if patient flares.

Treat:
Abdomen treatment (begin 15 or 60 Hz).
Pad extremities.
Scalp treatment (begin at 60 Hz or if hypersensitive, 15 Hz).
Homeostatic points/nerve points 1-12 (increase frequency . . . 180 Hz, 240 Hz if tolerated).

Day 5-14: At this point you will be addressing symptom changes or lack of them. The goal is the following:
cycle through the 24 Homeostatic/nerve points 2-3 times in 14 visits (as you did in Day 2 & 3).
organ points can reduce pain and inflammation significantly, so they need to be addressed at least every 2-3 visits.
cycle through abdomen treatment at least 3×/week.
alternate scalp and face treatment daily.
treat reflexively according to their new “old-reversal” pains (Hering's Law)
Manual treatment-Begin manual treatment to reflexive body part and once tolerated go directly on the involved body part briefly starting with a low frequency and gradually increasing as tolerated. Once direct treatment is tolerated on the involved body part for 5 minutes then progress to dynamic treatment.
Dynamic treatment.

Additional Options: Address any skin changes with each treatment. If bruising appears, treat directly over bruise pad red areas, treat rashes with pad or direct. Be aware that if the patient is severe and chronic you may not be able to treat directly on the involved body part until after the first set of 14 visits. This is not uncommon for chronic patients. If the treatment is working on healing internally or other areas of the body then direct treatment to the involved body part will only flare it. (Hering's Law: the body heals from the top down and from the inside first then the outside i.e., joints. Therefore, if the involved body part is the foot, it will be one of the last areas to get relief.)

Change frequencies with each visit. In general, initial treatments start with low frequencies and gradually increase with each treatment as patient tolerates. Use cycles and variable frequencies with pads, use constant stimulation patterns with Homeostatic/nerve points, organ points, face and scalp.

Pay attention to the Point Stimulation numbers (yellow numbers) and record them for the next visit/s.

Pad the places where the numbers are high on the next visit as these are good places to treat.

Once you have cycled through the frequencies starting low on 15 Hz and working up to 360 Hz, then alternate frequency settings to avoid accommodation.

Use 480 Hz on acute injuries or “newer” pain. You should never start on this frequency on a chronic condition as it can flare the person.

Pay attention to how your patient responds to certain frequencies. Some patients do well with high frequencies and some do not—it will flare them or their skin is too sensitive. Some patients may never tolerate 180 Hz or higher.

Some people may not sleep if using high frequencies too often. Low frequencies are usually better for sleep.

During these treatments also address soft tissue problems, use myofascial release (MFR), stretching, joint mobility followed by InterX afterwards. Be very cautious to not initiate soft tissue work too soon. Inflammation must be lowered before soft tissue work or the patient usually flares. Treat at least 6 visits before soft tissue work. Begin only if symptoms are reducing.

Address gut issues as needed changing supplements as needed.

Sleep must improve before permanent pain reduction. See pad chart for sleep locations.

Be creative with pad locations, remembering to pad reflexively, address all the organ channels as much as possible, nerve root distribution according to dermatome chart.

Reinforce that function will improve before sustained pain reduction. This function includes: improved gut function, improved sleep, the desire to “do more”. The patient will be able to do more activities at home even if the pain seems the same. They will usually notice the ability to recover from activity better than before. The intensity will be reduced but the pain may still be present, but their activities will slowly increase especially as sleep and gut functions improve.

Day 12-14: patient may be getting close to overstimulation by now. You will need to lower the frequency on the last 2 visits before break. If the patient is “over stimulated”, they will feel the InterX stimulation to be very strong at lower setting and they will need a lower intensity. They also might notice less times of relief post treatment (for example, normally having several hours to days of relief, they might only a few hours).

After 14 visits/break:
The patient will break from treatment for 7 days.
If the patient has a device, they may treat their face and organ point if needed and stay off of the scalp, Homeostatic points and abdomen. They can treat on their places of pain and opposite or reflexive points.
Patients are encouraged to not treat at all and let the body rest from treatment. It is during the break that the body mends and repairs. Often the patient will get good results until day 6 or 7 of the break then the pain often will return, sometimes just as bad as when they started. Warn them of this so they are not surprised. The pain will subside within the first one or two visits after the break. Often the pain does not return or just mildly.

Depending on the outcome of the 1^{st} set of treatments, it may be necessary to provide a 2^{nd} series of treatments (Day 1-10).

Day 1: Review the progress/set backs with your patient. Take notice of internal changes of improved sleep and improved gut and the newest pain that the patient had on Day 1 of Interx. Review supplements, Gut issues, sleep, medications, stress, anxiety, lifestyle changes.

Treat:
Remap the spine and face. Use a higher frequency: 180 Hz or 240 Hz.
If the person returns flared and can only tolerate low frequencies then use 60 Hz. Record your findings and compare to initial mapping for reference.
Begin Homeostatic points/nerve points if time permits.
Pad the extremities according to points of pain, inflammation, pad reflexively.

Day 2: Gut and sleep issues should be improving. If sleep is poor, stay on lower frequencies.

Treat:
Continue treating Homeostatic points (use higher frequencies: 240 Hz, 360 Hz).
Organ points (use higher frequencies, mix it up).
Scalp treatment if time permits (use higher frequencies above 180 Hz).
Dynamic application at the end of each treatment (see addendum).
May want to treat direct over the point of pain, point of stiffness.
Pad reflexive places while incorporating stretches.

Day 3: Always assess sleep, gut, function, stress—get counseling if necessary.

Treat: (use frequency of choice per tolerance . . . make sure you are changing the frequencies each visit).
Pad the spine, extremities.
Treat scalp.
Treat abdomen.
Treat face if time permits.
Treat directly if tolerated.

Day 4:

Treat:
Treat back points.
Back of the scalp well if you couldn't before (if patient was lying down previous days).
Organ points.
Dynamic treatment, adding any manual, PT or OT interventions that work well usually by this time.

Day 5-10: If the patient's symptoms continue to exist, then continue cycling through the protocols:

Treat:
24 Homeostatic points.
organ points.
Abdomen treatment.
Alternate scalp and face treatment.
Direct or indirect treatment to involved body part as patient tolerates and focus on reflexive treatment manually and dynamic treatment.

Alternate frequencies each day of treatment. If symptoms persist at this point the patient usually has moderate gut, sleep and/or stress issues that need to be corrected in order for permanent relief and treatment success. There are many reasons we have seen clinically:
Patient has a food intolerance that they continue to ingest keeping them inflamed.
Too much sugar, wheat or dairy, or high histamine food (must do supplements or low histamine diet).
Patient does an exercise or over exercises that inflames the involved area or keeps the body inflamed from too much exercises.
Lifestyle is too stressful, doesn't go to bed early enough and not getting 7-8 hours of sleep.

Day 9-10: Patient will be getting close to over stimulation, use low frequencies on last days. After day 10 the patient will break for 2 weeks. After 10 days of treatment/during 2 week break:
the patient will break for 2 weeks/14 days.
If they have a device they can treat their face, organ points, and point of pain if needed.
Again, if the patient feels good, they do not treat and let the body heal.
Focus on sleep, minimal exercise, healthy diet.
Patient should actually feel much better during the 2 week break, there are always exceptions if the patient keeps a stressful lifestyle or eats poorly.

Depending on the outcome of the 1st and 2nd set of treatments, it may be necessary to provide a 3rd Series of InterX Treatments (Days 1-6).

Day 1: Assess patient's status/progress and record changes in sleep, GUT/GI, pain, inflammation, function. Address supplements, home device use, sleep, Gut, stress, lifestyle.

Treat:
Remap the spine using frequency (60, 180, 240 or 360 Hz).
Face.
If time permits, treat organ points.

Day 2:

Treat:
Homeostatic/nerve points 1-12 (180, 240, 360 Hz).
Scalp treatment (180, 240, 360 Hz).
Treat directly and assess point of pain.
Dynamically or direct if tolerated.

Day 3:

Treat:
abdomen (use 60, 180, 240 or 360 Hz).
organ points.
direct or reflexive treatment or dynamic.

Day 4:

Treat:
back points.
back of scalp.
direct on point of pain or indirect.
Incorporate dynamic application.

Day 5:

Treat:
Face (use lower frequencies).
Review recommendations for home treatment if the patient has a device or determine if the patient needs to keep coming in OR if the patient wants to have a device for home use to self treat.
organ points.
abdomen at lower setting as the body will be beginning to plateau.

Day 6:

Treat:
scalp (low frequency).
point of pain (low frequency). dynamically with stretching or exercise.
Discuss with patient any recommendations you may have for exercise, stretching and recommend patient having a home InterX unit to self treat. You will focus on training them at this time and schedule a follow up in 1 month.

Dynamic Application. Passive Mobility, Contraction, Resistance and/or Stretch Caution: Use clinical judgment when considering Dynamic techniques. Elicit pain by position or motion. Target treatment for 30 seconds on each pain site while repeating pain position or motion. The pain may move, target new pain sites as time allows. Advance the exercise, if appropriate.

### Example 2

### Ankylosing Spondylitis

Diagnosis: Ankylosing Spondylitis—Age: 17. Patient had chronic pain for about ten years. patient have ankylosing spondylitis which caused me not to be able to go to school or hang out with friends and being a teenager. This was tough. Patient was very skeptical, thinking, if not even Remicade and heavy pain killers can help, how can this method?

Patient proceeded to go to the appointment, not really thinking much would come out of it. Patient's back was treated, which are original location for pain, within minutes Patient felt pain free. Patient has been undergoing treatment for one year, and no longer takes any medication. Before treatment patient was on: Remicade infusions, Methotrexate, Celebrex, patient took Vicodin for pain, as well as Tylenol 3. Patient is no longer taking any of those medications, stopped with the Remicade infusions. Patient is now a full time student and working full time as well.

### Example 3

### Back Pain

Diagnosis Back Pain—Age: 29. Patient is a 29 years old and experienced back pain that kept patient from doing or enjoying every-day activities like sleeping and walking. Patient would wonder each night before going to bed about whether or not he/she would be able to put on shoes in the morning. Patient tried physical therapy, visited a chiropractor, and took pain killers and muscle relaxers. These were all temporary fixes and patient's condition never improved. After being treated patient can now enjoy golf, basketball, and working in the yard with no back pain and my sleeping has drastically improved.

Patient 46, having bulging disc at C6/C7, as well as arthritis, bone spurs, and foraminal narrowing. Patient awoke with pain in neck that traveled down, just inside left shoulder blade. Patient thought it was a trapezius muscle pulled during sleep. Patient took some ibuprofen, and went on with the day. Of course, the pain got worse, and quickly reached a full 10/10. Over the next few weeks, patient's left triceps muscles stopped flexing, and became flat. Patient was diagnosed: bulging disc at C6/C7, as well as arthritis, bone spurs, and foraminal narrowing. Over the next two years, patient tried everything: physical therapy, which made the pain worse; acupuncture, with limited, short-lived relief. Two surgeons recommended invasive surgery.

Some relief came with regular visits to a chiropractor, who was able to reestablish nerve flow to the left triceps. However, the pain stubbornly remained, staying around 5 out of 10, or more.

Patient was skeptical of electrode treatment but hurting enough to give it a try. An initial 14 in-office treatments were recommended, in addition to monthly maintenance treatments at home. Patient was able to get good results after only 11 office visits. Patient reports that the difference after treatment, is like night and day. Today, 3 months after the initial treatments, patient pain is rarely above a 2 out of 10. And there are actually some days with NO pain. Patient has avoided surgery, and have no plans to do so.

### Example 4

### C6-7 Bulging Discs

Diagnosis: C6-7 Bulging Discs—Age: 52. After visits with two different neurosurgeons patient was advised that surgery may be the only correction for my neck pain and hand numbness. Patient completed a series of electrode treatments noticing neck pain relief after five sessions. The numbness in patient's hand subsided after eight treatments and was completely gone after eleven. With the incorporation of a home treatment system patient has been able to eliminate the numbness in hand and control neck pain.

Prior to InterX electrode therapy patient was unable drive without tilting their head forward to look over glasses nor was able to ride a bike, play racquetball or participate in other activities. After InterX electrode treatment patient no longer has any limitations or require pain medication.

### Example 5

### Back Pain, Sciatica Pain

Back Pain, Sciatica Pain—Age: 70. Before referral for InterX electrode treatment patient tried other forms of pain management that ended in failure. Patient was diagnosed with arthritis in lumbar spine that sent pain down left hip and leg. Patient tried several steroid injections with inconsistent results. Patient also tried chiropractic treatments with no lasting benefits. Patient then relied on prescription and OTC pain medications before InterX electrode treatment. Patient started with treatment 3 times per week and gradually reduced the treatments as the pain reduced. Received a home unit and began treating myself at home. Today patient experiences only infrequent pain which is eliminated by self-application with the home unit. Patient no longer uses pain relievers of any kind and at 70 years old does not have to suffer from arthritis pain as a result of InterX electrode treatment.

### Example 6

### Back, Sciatica Pain

Back, Sciatica Pain—Age: 63. Patient injuries date back to 1968 from two accidents in construction work. Patient had nerve damage in right foot and upper back. The foot injury generated continuing pain that patient was generally desensitized to but sometimes elevated to be severe pain. Patient back injury limited certain activities but in both cases, nothing was observable to friends or family.

Patient tripped and fell twisting body. Patient did not have any scrapes or bruises but this fall aggravated past injuries with the following symptoms: Sharp severe sciatic nerve pain in hip; the origin was a myelogram associated with back injury. Sharp severe nerve pain in the right knee, calf and foot. Patient could not tolerate walking any more than 50 ft. or stand more than 5 minutes; the pain was severe and had no stamina. Pain even continued with inactivity; there was constant discomfort. Frequent episodes of extreme nausea. General sense of nervousness that elevated when patient was stressed.

After approximately 10 InterX therapy sessions patient began to improve. The sessions continued along and along with home use of the InterX device, patient was close to normal after 15 or 16 therapy sessions. The only medication patient took during the recovery was Tylenol. Patient is now in very good condition and continues to use the InterX device at home on a regular as needed basis.

InterX Results: After 12 InterX electrode treatments, patient reported 2+ prescriptions medications to none, minimal to no pain or discomfort (pain level of 8 decreased to 0 out of 10), mental fog clear, resumed active lifestyle and returned to school. As of 2018, patient remains 100% Opioid Free since 2006 InterX electrode treatment (12 years).

Prior Results: Patient reported these symptoms, medications, treatments and a pain level of 8 prior to InterX treatment:
Symptoms: Debilitating back pain at the T11-12 joint, Extreme Lethargy. Medication: Hydrocodone, Tramadol.

Treatment: Injections, Nerve Ablation, Physical Therapy, Massage, Back Brace.

### Example 7

### Ehlers Danlos Syndrome, Hypermobility Type

Subject Age: 29, Profession/education: Pediatric Nurse Practitioner/MSN, Lifestyle/Hobbies: Bike Riding, running, playing with dog, reading, photography.

Condition/Symptoms: Ehlers Danlos Syndrome, Hypermobility Type/Currently no symptoms, but formerly had debilitating back pain at the T11-12 joint on the right side.

Previous Treatments: When patient was in high school had debilitating back pain that all but ended active lifestyle. Patient had pain from the moment he/she woke up in the morning until patient went to bed a night, which was then aggravated by any activity, sitting and walking.

Patient had even tried two rounds of nerve ablation, in which they burned off the nerve endings at the joint patient's back that caused the pain, but the pain still returned. Patient was then told the only way to break the pain cycle was to medicate with high-dose, narcotic pain medicine. Patient had many months of taking medicine like hydrocodone, tramadol and others, which this dulled the pain, but it also made patient so sleepy he/she wasn't able to function in day to day life. Patient couldn't go to school, or even think about doing homework, playing college basketball had long been forgotten. Along with all of this patient has also tried massage, ice, rest, back braces, physical therapy & muscle relaxers.

InterX electrode treatment were able to break the pain cycle, even with just one treatment. All these years later. Patient had tried every pain management strategy, and after just one treatment with the InterX electrode treatment broke the pain cycle. Patient was able to stop taking prescription pain medicine.

Patient remains pain free, having completed graduate school and living a very active lifestyle after InterX electrode treatment. InterX electrode treatment eliminated battling constant, chronic pain.

InterX Results: After 12 InterX treatments, patient reported 2+ prescriptions medications to none, minimal to no pain or discomfort (pain level of 8 decreased to 0 out of 10), mental fog clear, resumed active lifestyle and returned to school. As of 2018, patient remains 100% Opioid Free since 2006 InterX treatment (12 years).

Prior Results: Patient reported these symptoms, medications, treatments and a pain level of 8 prior to InterX treatment:

Symptoms: Debilitating back pain at the T11-12 joint, Extreme Lethargy. Medication: Hydrocodone, Tramadol.

Treatment: Injections, Nerve Ablation, Physical Therapy, Massage, Back Brace.

### Example 8

### Fibromyalgia

Fibromyalgia—Age: 19. At age 16 patient pain began and by 18, patient was physically disabled. Patient was diagnosed with fibromyalgia and the doctors said there was no actual “cure” for it. Patient couldn't bend down to play legos on the floor with nephew because the intensity of knee pain was immeasurably painful. On top of that, patient had to have their mom drive because patient couldn't even bend wrists or ankles without feeling a sharp knife-like pain. Patient went to countless doctors and took almost every medication out there for the pain—but still, nothing seemed to cure the pain because it would just come right back. Patient felt so hopeless because summer was almost over, and patient was going away to college and wouldn't have the support back at home to help me if patient to experience the same crippling pain.

Patient underwent InterX electrode treatment, and was like a new person. After initial treatment patient was able to have an entire night of sleep without waking up from the pain due to pain in wrist/ankles. Patient continued the treatment for 2 more weeks and was able to return to college with the pain gone. Patient was able to bend knees and played ultimate frisbee in front of dorm, and go cliff jumping without experiencing excruciating pain. Upon flare-ups of pain patient follows-up with InterX home treatment that immediately takes away the pain by the next day. Patient continues live a normal, pain free life.

### Example 9

### Fibromyalgia and Rheumatoid Arthritis

Fibromyalgia and Rheumatoid Arthritis—Age: 50, Patient had 24/7 excruciating pain in the upper neck and across the right shoulder for 3 years after an auto accident. Patient was mentally preparing to manage a life of constant pain. Patient already had rheumatoid arthritis for 6 years and was now diagnosed with fibromyalgia after the accident.

Patient has InterX electrode treatment and after 7 therapy sessions had significant reduction of pain. Patient was able to stop taking Lyrica, hydrocodone and anti-inflammatories. Patient brain fog became clear, sleeping improved and energy levels returned. Patient now uses a home device and can manage occasional flare ups.

Fibromyalgia and Rheumatoid Arthritis—Age: 27. 27 years old patient with special needs due to an underlying neurological condition. Patient has had over 70 surgical procedures in the past including a complete hysterectomy. Not long after that, patient developed chronic headaches. Patient saw neurologists, headache specialists, pain management doctors, acupuncturists and chiropractors who tried just about everything they could think of to help her headaches . . . all without success. Patient was on a wide variety of narcotics, and many other potent medications to try to control the headaches but to no avail. Patient found pleasure in nothing and was only remotely comfortable when lying down in a dark room.

Patient received twice a week InterX electrode treatment and slowly but surely the pain levels subsided. Patient was slowly taken off of the various medications she was taking (which were causing difficult side effects), and the pain kept improving.

Patient also had a lot of accumulated toxins in her system as a result of all the medication she was taking . . . which limited the efficacy of other medications and supplements she was taking. After this course of treatment, Patient's blood work showed dramatic improvements in many areas, such as her thyroid which has allowed her to almost completely eliminate this medication. Prior blood work had shown significant parasitic activity and limited bowel motility. Again, the blood work after patient's course of InterX electrode therapy demonstrated a notable decrease in parasites while Megan's bowels returned to normal function.

Patient is virtually pain free now . . . and when she does get a headache, she uses an InterX device at home and within minutes her headache is gone. This has transformed Patient's life.

### Example 10

### Severe Headaches

Severe Headaches due to car accident head trauma—Age: 56. Following a car accident in December 2008, patient began to have severe headaches due to whip lash and head trauma. Patient sought out remedies for headaches over the next eighteen months including chiropractors, muscle relaxers and pain pills, two shots to block the nerves in the head, acupuncture, and Botox to the forehead and neck.

Patient initiated InterX electrode treatment and began to see improvement in the headaches. Within two weeks of treatment, patient was able to stop all pain medication except for the occasional aspirin.

After completion of the InterX electrode treatments, headaches are mild and infrequent. Patient uses a device at home about once a week to keep the headaches under control. Patient would recommend InterX Therapy to anyone suffering from migraine or other type headaches.

### Example 11

### Femoral Nerve Damage

Femoral Nerve Damage—Age: 56. InterX electrode therapy was used to treat femoral nerve damage. Patient now visits the center a couple times a year and uses an InterX home unit to treat myself between visits.

During a medical procedure, patient sustained permanent damage to femoral nerve. At first, the damage resulted in what patient would describe to be an inconvenience but within a short time patient had, at times, unbearable pain. The weakness in patient' leg resulted in being hardly able to walk and to go any distance and required that patient begin use of a wheel chair.

Over a period of two to three years patient saw many doctors too numerous to list from neurologists, pain management, orthopedics'with no significant improvement other than taking pain pills to relieve the discomfort.

Patient self-treated with InterX electrode therapy on loan from a medical professional and within days could feel measurable improvement. Patient then opted for full InterX electrode therapy and at first was very cautious as patient had been disappointed before with other treatments. After the initial treatments, and following the suggested treatment regime from the center, the results were unbelievably significant. With time patient had significant relief which allowed patient to lead a normal life. While the damage in patient's leg is permanent, Interx electrode therapy has done wonders for patient.

### Example 12

### Chronic Nerve Inflammation of Sacrum and Coccyx

Chronic Nerve Inflammation of Sacrum and Coccyx—Age: 67. Registered Nurse working for a major insurance company, spent last 10 years either sitting in a chair all day at a computer or flying across the country to deliver training to other nurses who will interact with consumers telephonically. Traveling and meeting new people was my favorite part of the job. But all of that came to an end due to intermittent back spasms from prolonged sitting. A week-long virtual seminar almost brought patient's professional and personal life to an abrupt end. Pain was so severe, patient thought he/she would have to go out on medical leave or disability.

Patient has been training nurses on how to coach consumers who were experiencing recurring back pain or making a decision about whether to consider back surgery, as such, patient thought he/she knew just what to do. While continuing to travel 50% of the time or more, patient undertook six months of chiropractic treatment supplemented by massage therapy. Despite cold laser, ultrasound, heat and adjustments, the pain in sacrum and coccyx kept returning within two days of treatment.

Patient was successfully coping with the pain by wearing a TENS unit all day and all night, but developed an allergic reaction to the adhesive on the electrodes.

At that point, patient visited primary care physician and requested a referral to a back clinic and prescriptions for pain, including 5 mg of hydrocodone and 100 to 200 mg of Lyrica. Fibromyalgia was under consideration as a diagnosis, so patient had a complete neurological workup, including a nerve conduction velocity test that ruled out diabetic neuropathy or any neurological disease. Patient asked for an orthopedist who worked with patients requesting conservative therapy, and got an MRI and a prescription for 7.5 mg of Mobic. When patient visited the back clinic, patient was unable to sit on any surface, no matter how soft. To avoid 8/10 pain in the sacral area, patient had to be lying down on a therapy table or standing up. The MRI results revealed no disc compression or significant pathology. It found a rotated sacrum and Tarlov cysts located on the sacrum. PT was focused on building core strength and attempting to rotate the sacrum to relieve muscular tetany in the left IT band. At that point, patient activity limitations were no sitting for more than 10 minutes, no squatting, kneeling, stairs or crawling; no lifting of more than 10 pounds. Because this effectively meant patient could not perform job duties, patient was moved from the training delivery job to a curriculum development position.

Physical therapy and Mobic relieved about 50 to 60% of pain, but patient was still having frequent episodes of recurring pain that none of the medication could manage. Near the end of the last month of therapy, pain was almost completely gone.

InterX electrode therapy returned patient's ability to walk, relieved patient of pseudo-seizures and made patient fully functional again. Patient now maintains using home therapy device.

Interx electrode therapy included the use of multiple Interx devices for treatment all over the body simultaneously. As expected, for the first 10 visits, pain appeared to be actually getting worse, however, within a week or two, patient had increased flexibility, fewer episodes of increased pain, had an improved ability to fall asleep and stay asleep and relief from Irritable Bowel Syndrome. At that point, patient was in for the duration and bought my own device.

After treatment regimen, patient was able to weed flower gardens to loosen tetany in left hip; pain episodes were rarely more than 4/10, and patient could sit for more than 10 minutes. Some days patient could lift left leg lifted and turned enough to see the bottom of foot.

One-month later patient was able to sit on a hard wooden bench for an hour without any pain. Patient could sit in a softer office chair all day, fall asleep with ease anytime and anywhere. Patient was then able to resume additional physical activity such as skipping, doing Samba moves and eventually cross one leg over the other leg, left over right. The worst pain, usually brought about by cold, created only 2/10 pain in knees, one ankle and the sacrum. Patient was able to discontinue the Mobic, the Hydrocodone and the Desipramine for IBS.

Using a home unit, patient was able to renew air travel, lift heavy bags and twisting them in a car trunk seemed to provoke the worst discomfort, and treated with the home Interx unit helped to return patient to a pain free state. Once the big climate shifts settle down near the end of February, patient will be attempting to wean myself off the Lyrica and plan to be taking no drugs for pain at all.

As a clinician, patient had severe reservations about technologies that did not standard clinical evidence that they were efficacious in the majority of cases. Patient has reconsidered this position. After Interx electrode treatment, patient is now able attend movies or go out to eat without carrying a bulky memory foam pillow to sit on.

Patient states that, “this is a non-invasive, highly effective treatment for pain that also solves problems with function and with organs. It is my belief that the only thing that pulled me back from having to go out on disability is the 24 InterX treatments twice a week.”

### Example 13

### Neuropathy (Chemotherapy Induced)

Neuropathy (Chemotherapy Induced)—Age: 55. At the age of 55, patient was diagnosed with stage 3 Colon Cancer. Successful surgery was followed by 12 sessions of adjunctive chemotherapy (Folfox). While the chemotherapy was less brutal than some, it still came with its side effects, leaving patient with chemotherapy-induced peripheral neuropathy (CIPN) in hands and feet. Patient's hands became intolerant to the cold, hypersensitive finger tips and loss of fine motor skills. The symptoms in patient's feet included: numb toes, hypersensitive soles, intolerance to cold, partial loss of balance, minimal flexibility and edema.

Patient underwent 14 InterX electrode treatments in a 4 week period and the swelling (edema) was completely eliminated. The other symptoms patient was experiencing in the feet would lessen for a few minutes to hours but then return. Patient's hands responded slowly but eventually returned to 100% normal within 5 months from initial session.

Patient returned for InterX electrode treatments one year later with the previous symptoms in the feet. After the 4th day of treatment, patient had a breakthrough day with the feeling returning to the toes. Patient completed another 12 treatments of therapy and had full restoration of flexibility and a normal sense of balance. The remaining symptoms slowly abated and feet are now 90% normal. Patient has been released to work after being on medical leave for approximately 2 years.

### Example 14

### Phantom Limb Pain

Phantom Limb Pain—Age: 63. Amputated patient started taking Hydrocodone 10/325 every 6 hours, 2400 MG of Neurontin a day, 60 MG of Cymbalta for the pain and emotional stress and wearing a 75 mcg Fentanyl Transdermal Patch. In 2012, patient lost their balance and fell; breaking a hip on the same side as the amputation. Patient now has phantom pain, hip pain, back pain (from a previous surgery), severe rheumatoid arthritis pain along with stomach problems from all the medications.

Patient worked for an electric company doing nondestructive testing on machines and sensors for industrial applications requiring surface and sub-surface crack detection and metal sorting using such tests as ultrasonic, magnetic-particle, liquid penetrate, radiographic, remote visual inspection (RVI) and eddy-current testing to meet the critical requirements for quality, productivity and safety.

After multiple injuries and surgeries on his left knee he was forced to take early disability. Patient loved his job and working with his co-workers in the field. His work was like therapy for his pain, which didn't take it away, but gave him something to distract his mind from it so much.

Patient's medical history includes many surgeries: Back Surgery—no infection, 2 meniscus tear surgeries on left knee—no infection, 2000 Shoulder surgery for torn rotator cuff—no infection, 2000 Gallbladder Surgery—no infection, 2006 Renal Cell Carcinoma, Remove part of kidney—no infection, 3 total knee replacements on left knee—infections, 10 surgical revisions including muscle flap surgery on left knee—infections, 30 hyperbaric oxygen treatments for attempted healing of muscle flap surgery, and 2012 Hip Replacement—no infection.

After 3 total knee replacements and 12 surgical procedures on the same knee, patient continued to have a Staph infection from the first surgery.

Patient's leg was loose around the prosthesis, it was constantly swollen, hot to touch, red and painful. Patient returned repeatedly to surgeon and would either be scheduled for another surgery, physical therapy or told nothing was wrong. Patient went to new doctor, an oncologist, and he ran a nuclear scan on leg and was told infection was so bad patient was going to have to make a decision to “lose a limb” or “lose my life”. Patient consulted with surgeon and infectious disease doctor and they told patient the leg was not infected.

Patient went to Mayo clinic and they confirmed oncology doctor's findings. The MRSA and Pseudomonas was so bad it was close to going septic spreading all thru my body killing my organs. Patient consented to an above the knee amputation. After the amputation patient's body started to heal and patient was feeling better, but patient developed phantom pain which was worse than the pain patient had before the amputation; but patient was alive and body was healing.

The additional pain was almost unbearable, patient started taking Hydrocodone 10/325 every 6 hours, 2400 MG of Neurontin a day, 60 MG of Cymbalta for the pain and emotional stress and wearing a 75 mcg Fentanyl Transdermal Patch.

After just 3 weeks of InterX electrode therapy patient has been pain free for up to two days at a time from hip pain, back pain, phantom pain, stomach problems, headaches and pain from my Rheumatoid Arthritis. Patient has weaned off the Cymbalta and was able to cut down on my pain medication. Patient's thinking is clearer and no longer has “foggy” feeling all the time.

### Example 15

### POTS Postural Orthostatic Tachycardia Syndrome, EDS

POTS Postural Orthostatic Tachycardia Syndrome, EDS. Patient, 17, has RSD/CRPS. After receiving 14 InterX treatments at the InterX Therapy and Research Center, patient stated, “I went from 5 prescription medications to none, minimal to no pain or discomfort [pain levels of 7 to 0 out of 10], digestive issues resolved, 0-1 migraine per month and decreased depression, anxiety and better sleep.”

Patient was diagnosed after removing a screw in ankle from a fall while rock climbing. The screw was to stay in ankle for a full year. After 7 months, patient was in unbearable pain surgeons thought it was because the screw may have been rubbing against the bone every time her ankle moved. The decision was made to remove the screw. When patient came home from surgery that removed the screw, ankle swelled larger than a grapefruit, the top of her foot and ankle had discoloring, and lesions formed. It was assumed swelling was from the surgery, the discoloration was just bruising, but the lesions looked like a scar from deep cuts.

For three straight days, patient did not sleep because the pain was so severe. We were told that removing appliances is a lot more painful than placing them. The pain medication did not even come close to taking the pain away.

When patient went in for a follow up appointment, everything looked good as to where the screw was concerned. The surgeon thought the discoloring was bruising, and the lesions can be caused from the swelling patient incurred. Patient was released to play sports as soon as she felt she could. A couple weeks after patient's follow up appointment, patient started to have painful tingling in ankle and top of foot. It was thought that maybe when the screw was removed, a nerve may have been touched and may have needed more time to heal. Patient continued to play soccer during this time.

The painful tingling continued and was getting worse each day. Patient returned to the surgeon's office to have this checked and was diagnosed with Reflex sympathetic dystrophy syndrome (RSD). Because RSD takes away mobility in the muscles, patient started physical therapy that week, for a full month, three times a week. As the weeks went by, the pain and tingling were getting worse. Now sharp pains were starting too. At this time, the decision was made to switch to aqua therapy so patient wouldn't have to bare their body weight while doing the exercises. Patient did this for a few weeks. It helped keep patient muscles mobile, but that was it.

After another visit to the surgeon's office the patient was referred to a pain specialist, and of course, was gave prescriptions for the pain. Which, since RSD patients can't absorb medications, did not help with the pain patient was having, but would knock patient out cold for a bit.

By this time, patient was losing her functions. Patient was homeschooled and had to stop doing schoolwork halfway through the year. Patient no longer slept and would wake up from the pain. At this time pain level was 9-10 and patient had a very high pain threshold. Further, patient was unable to wear socks or shoes because of the pain.

The pain med specialist thought patient should try blocks that injected into the spine, hoping it would calm the nervous system. Patient began nerve block treatment, but it lasted for a few hours, hoping that with each additional block it would last longer and longer and that the blocks would provide relief with a hope of achieving remission.

Patient had one block a week for four weeks. The first one did nothing. The second nothing, the third worked and gave patient enough relief to attempt sports, but the block wore off and almost instantly the pain returned. After the fourth block, patient woke up numb and tingling on her left side. They thought she had a stroke and blocks were terminated. When patient started InterX electrode therapy, foot and ankle pain were at their worst.

When patient walked, the foot dragged behind and could not have any weight on it. To go up or down the stairs, it was necessary to use rails as a crutch. The pain to have anything touch patient was unbearable, with a pain level at a constant 9-10.

After about a month of InterX electrode, patient noticed something that isn't a big deal to most people, but was a huge milestone, patient was able to walk down a small flight of without holding onto the railing. Eventually, patient's foot and ankle were to the point that patient could tolerate treatment directly on them. Patient's foot stopped dragging, and walking got better.

Patient was able to resume schoolwork, no longer felt cold most of the time, and all function returned. The last to return was sleep.

Six months after starting Inter X electrode treatment, patient was released with a pain level at about a 3. Patient was able to continue running, eating, studying, sleeping. After completing her treatment, patient continues use of home InterX treatment, and within 4-6 weeks after being released, was in complete remission and even playing soccer.

### Example 16

### Complex Regional Pain Syndrome (CRPS)/Reflex Sympathetic Dystrophy Syndrome (RSD)

Complex regional pain syndrome (CRPS)/Reflex sympathetic dystrophy syndrome (RSD). Patient—Age: 27. Multiple doctors and PTs gave up on patient after their first visit, and the drugs never did anything for patient besides side effects, so patient didn't continue taking them. It took 10 months, 2 more ineffective injections, 2 more types of PT, another chiropractor, another 3 types of useless drugs, 3 more MRIs showing minimal change in a disc, another “normal” nerve conduction study+EMG and 6 more doctors with worsening of pain and nerve symptoms, before patient finally found a surgeon willing to help.

Patient had a successful minimally invasive laminectomy. The surgery removed the tugging/irritating pain on the nerve that patient previously got whenever patient moved, but tingling, nerve pains, and numbness in leg and hip persisted. Patient's PT finally diagnosed patient with RSD.

Upon the first InterX electrode therapy, patient almost immediately started to have first real reversals in nerve symptoms, patient started to be able to maintain posture while decreasing time spent doing PT exercises, the SI joint pain that patient had struggled with for years disappeared entirely, flare-ups became less frequent, and patient could bend their back enough to sleep on their stomach for the first time in almost 8 years!

After spending a third of their life in intense pain, patient had forgotten what a “normal” leg feet like. Patient often realized after a treatment that a pain was gone, in fact, patient didn't know the pain was there to begin with because it had been there so long. Tingling decreased through the whole leg and foot, the leg no longer constantly had a heavy/achy/tired feeling that makes it hard to stand or walk, hip feels stronger and more stable, and nausea is improving. Patient is no longer constantly completely stressed out, overwhelmed, and exhausted, sleep is improving and when patient does wake up at night, patient no longer has horrible hip pain that makes patient want to rip the leg out of its socket.

The InterX is not masking the pain or even just treating symptoms; it feels like patient's body is actually permanently healing for the first time since the original injury almost 9 years earlier.

After spending 7-8 years unable to do much with their lower body, patient's legs had become noticeably weaker, but patient is finally starting to feel like muscles are improving again and legs and hip feel stronger and more functional than they have in years.

### Example 17

### RSD: Ligament Injury

RSD: Ligament injury—Age: 45. Patient a simple ligament injury in one foot which ended up progressing into unexplainable neuropathic pain in both feet. Patient had difficulty being on their feet for any length of time, walking or standing. It took two years to get a useful diagnosis after having gone to many orthopedic specialists, podiatrists, physical therapists, other types of doctors (rheumatologist, endocrinologist, etc.), and various alternative medicine practitioners (chiropractors, etc.).

A neurologist finally gave patient a tentative diagnosis of Chronic Regional Pain Syndrome. The disorder is typically known as being incurable and degenerative. The treatments that are recommended are not curative but only temporarily helpful if they are even helpful at all. Patient was unwilling to try the nerve drugs, opioids, nerve blocks, or other invasive procedures because patient didn't want to do anything that would or could cause other problems in their body just for possible temporary relief. So, patient finally had a diagnosis but patient didn't know what to do about it or whether to have hope that it would ever get any better.

Patient initiated InterX electrode therapy. After the initial treatment, patient had maintenance treatments, and has been on InterX therapy for over a year. It has been the only thing that has made a difference in patient's pain level. From the very first appointment, Janice gave me hope that healing is possible.

### Example 18

### Scoliosis, Spondylosis, Neuropathy

Scoliosis, Spondylosis, Neuropathy. InterX Results: After 10 InterX treatments, patient reported 4 prescription medications to none, minimal to no pain or discomfort (pain level of 7 decreased to 0 to 1 out of 10), digestive issues resolved, chronic inflammation gone, reduced weight by 40 lbs., resumed active lifestyle and better sleep. As of 2018, patient remains 100% Opioid Free since 2017 InterX treatment (1 year). Prior results: Patient reported these symptoms, medications, treatments and a pain level of 7 prior to InterX treatment:
Symptoms: Opioid dependence, Restless Leg Syndrome, Severe Chronic Back and Neck Pain, Insomnia, Weight Gain. Medication: Hydrocodone, Zanaflex, Gabapentin, Horizant. Treatment: Epidural Steroid Injections, Rhizotomy.

Senior Project Manager lived an active lifestyle before experiencing severe chronic back pain due to scoliosis, spondylosis, neuropathy. Pain centers prescribed opioid medication, steroid injections and rhizatomies. Though effective at first, his body built up tolerances requiring increased dosages and side effects including signs of physiological opioid dependence. Inactive lifestyle due to inflammation was compounded by growing opioid dependence, weight gain from steroids, insomnia and restless leg syndrome due to the medication.

Since InterX treatment, patient has been free of medication, pain and symptoms. Overall inflammation is reduced, and patient has returned to an active lifestyle. Patient uses an InterX home device for the occasional upset stomach and other minor symptoms.

FIG. 14 is a table that includes additional examples of treatments of the present invention.

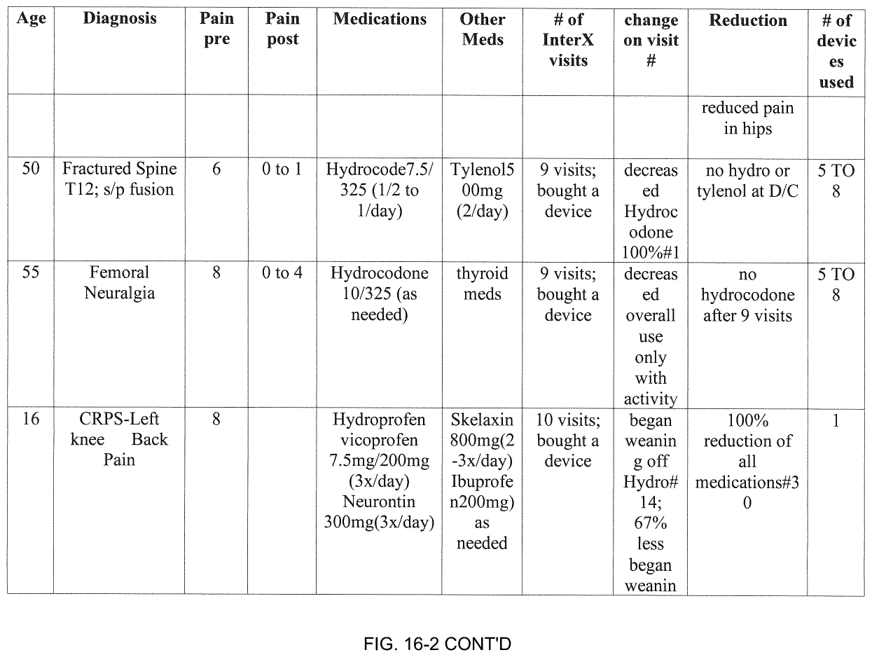

FIG. 15 is a table that shows examples of treatments of the present invention using a variety of arrays, multiple devices, comb electrode and a soft tissue electrode.

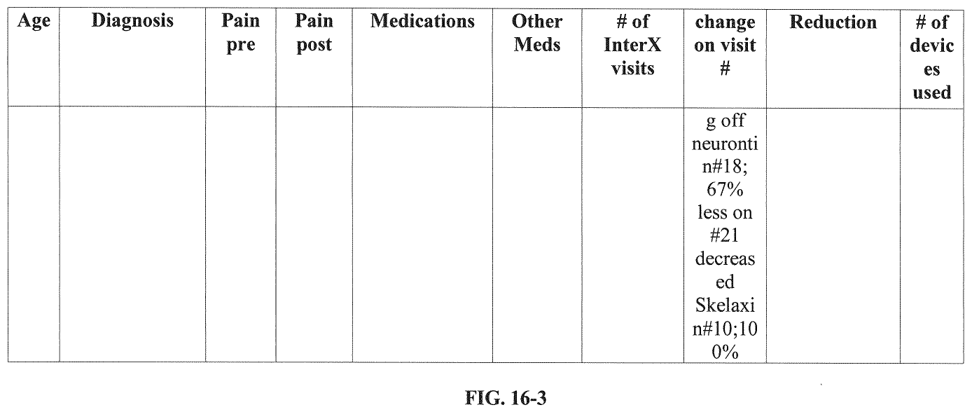

FIG. 16-1 to 16-3 are tables that includes additional examples of treatments of the present invention for opioid dependence and other dependencies.

The present inventors recognized that there is a significant lack of highly effective treatments for many chronic pain conditions, underscoring the urgent need for innovative therapeutic solutions. The InterX Full Body Stimulation Treatment represents a novel advancement in addressing this need. The simultaneous application of multiple electrical stimuli through an InterX neurostimulator device initiates a cascade of electrical and chemical processes, leading to nerve activation, signal transmission to the brain, and subsequent interpretation and response by the brain. This results in therapeutic effects, including pain relief and modulation of autonomic functions.

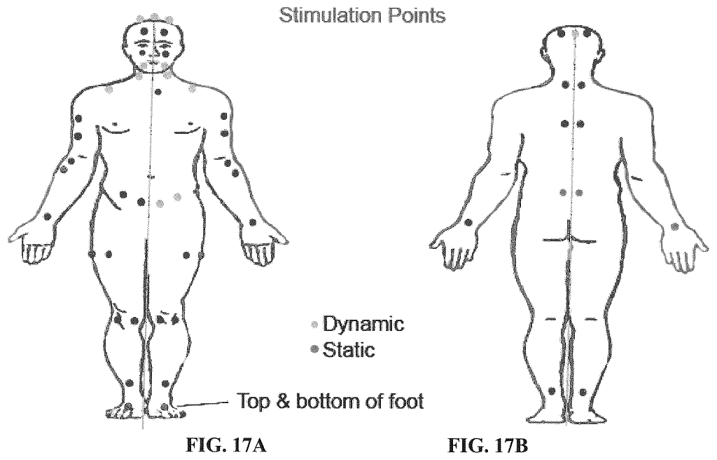

FIGS. 17A and 17B shows additional dynamic and static stimulation points used with the apparatus and methods of the present disclosure. FIG. 17A the frontal dynamic and static stimulation points and FIG. 17B the distal dynamic and static stimulation points.

Nerve pathways involved with InterX® Neurostimulation.

Foot to Brain. The following are stimulation points for use with the apparatus and methods of the present disclosure, including both dynamic and static stimulation points as shown in FIGS. 17A and 17B.

Pain Receptors Activation: When you experience stimulation in your foot, specialized nerve endings called nociceptors are activated. These receptors detect harmful stimuli, such as injury or extreme temperatures.

Transmission to Peripheral Nerves: The nociceptors send electrical signals through peripheral nerves. In the foot, these signals travel through the sciatic nerve, which is the largest nerve in the body.

Spinal Cord Entry: The sciatic nerve carries the pain signals up the leg and into the spinal cord through the dorsal root ganglia, located near the spinal cord.

Spinal Cord Processing: Once in the spinal cord, the signals are transmitted to neurons in the dorsal horn. Here, the signals are processed and can be modulated by other neurons, which can either amplify or dampen the pain sensation.

Ascending Pathways: The pain signals are then sent up the spinal cord through ascending pathways, primarily the spinothalamic tract. This tract carries the signals to the brainstem and then to the thalamus, which acts as a relay station.

Thalamus Relay: The thalamus processes the pain signals and forwards them to various parts of the brain, including the somatosensory cortex, which is responsible for perceiving and interpreting the pain.

Perception in the Brain: Finally, the somatosensory cortex, located in the parietal lobe of the brain, interprets the signals as pain, allowing you to consciously perceive the sensation and respond accordingly.

Thigh to Brain. The following are stimulation points for use with the apparatus and methods of the present disclosure, including both dynamic and static stimulation points as shown in FIGS. 17A and 17B.

Femoral Nerve: The journey begins with the femoral nerve, which is one of the largest nerves in the leg. It originates from the lumbar plexus, specifically from the L2 to L4 nerve roots.

The femoral nerve travels down the front of the thigh, providing motor and sensory functions.

Sensory Receptors: Sensory receptors in the skin and muscles of the thigh detect stimuli such as touch, pain, and temperature.

### Pathway to the Spinal Cord:

Femoral Nerve: Sensory signals travel through the femoral nerve.

Lumbar Plexus: These signals then pass through the lumbar plexus, a network of nerves in the lower back.

Spinal Cord: The sensory signals enter the spinal cord through the dorsal roots of the lumbar spinal nerves. They travel up the spinal cord to the brainstem.

### Brainstem and Thalamus:

Brainstem: The signals are relayed through the brainstem.

Thalamus: The thalamus acts as a relay station, directing the sensory information to the appropriate areas of the brain.

Cerebral Cortex: Finally, the sensory information reaches the somatosensory cortex in the parietal lobe of the brain. Here, the signals are processed and interpreted, allowing you to perceive sensations from your thigh.

Calf to Brain. The following are stimulation points for use with the apparatus and methods of the present disclosure, including both dynamic and static stimulation points as shown in FIGS. 17A and 17B.

Sciatic Nerve: The journey begins with the sciatic nerve, which is the largest nerve in the body. It originates from the lower spine and travels down the back of each leg.

Tibial Nerve: In the calf, the sciatic nerve splits into two branches: the tibial nerve and the common peroneal nerve. The tibial nerve continues down the back of the calf.

Sural Nerve: The sural nerve branches off from the tibial nerve and provides sensory information from the skin of the lower leg and foot.

Spinal Cord: The tibial nerve carries sensory signals from the calf up to the spinal cord. These signals travel through the lumbar and sacral plexuses, networks of nerves in the lower back and pelvis.

### Brainstem and Thalamus:

Brainstem: The sensory signals enter the spinal cord and travel up to the brainstem.

Thalamus: The signals are then relayed to the thalamus, which acts as a relay station for sensory information.

Cerebral Cortex: Finally, the sensory information reaches the somatosensory cortex in the brain, where it is processed and interpreted, allowing you to perceive sensations from your calf

Knee to Brain. The following are stimulation points for use with the apparatus and methods of the present disclosure, including both dynamic and static stimulation points as shown in FIGS. 17A and 17B.

Pain Receptors Activation: Pain in the knee activates nociceptors, which are specialized nerve endings that detect harmful stimuli.

Transmission to Peripheral Nerves: The nociceptors send electrical signals through peripheral nerves. In the knee, these signals travel through the femoral nerve and the sciatic nerve, depending on the exact location of the pain.

Spinal Cord Entry: These nerves carry the pain signals up the leg and into the spinal cord through the dorsal root ganglia, located near the spinal cord.

Spinal Cord Processing: Once in the spinal cord, the signals are transmitted to neurons in the dorsal horn. Here, the signals are processed and can be modulated by other neurons, which can either amplify or dampen the pain sensation.

Ascending Pathways: The pain signals are then sent up the spinal cord through ascending pathways, primarily the spinothalamic tract. This tract carries the signals to the brainstem and then to the thalamus, which acts as a relay station.

Thalamus Relay: The thalamus processes the pain signals and forwards them to various parts of the brain, including the somatosensory cortex, which is responsible for perceiving and interpreting the pain.

Perception in the Brain: Finally, the somatosensory cortex, located in the parietal lobe of the brain, interprets the signals as pain, allowing you to consciously perceive the sensation and respond accordingly.

Hip to Brain. The following are stimulation points for use with the apparatus and methods of the present disclosure, including both dynamic and static stimulation points as shown in FIGS. 17A and 17B.

Pain Receptors Activation: Pain in the hip activates nociceptors, which are specialized nerve endings that detect harmful stimuli.

Transmission to Peripheral Nerves: The nociceptors send electrical signals through peripheral nerves. In the hip, these signals travel through the sciatic nerve and the femoral nerve, depending on the exact location of the pain.

Spinal Cord Entry: These nerves carry the pain signals up the leg and into the spinal cord through the dorsal root ganglia, located near the spinal cord.

Spinal Cord Processing: Once in the spinal cord, the signals are transmitted to neurons in the dorsal horn. Here, the signals are processed and can be modulated by other neurons, which can either amplify or dampen the pain sensation.

Ascending Pathways: The pain signals are then sent up the spinal cord through ascending pathways, primarily the spinothalamic tract. This tract carries the signals to the brainstem and then to the thalamus, which acts as a relay station.

Thalamus Relay: The thalamus processes the pain signals and forwards them to various parts of the brain, including the somatosensory cortex, which is responsible for perceiving and interpreting the pain.

Perception in the Brain: Finally, the somatosensory cortex, located in the parietal lobe of the brain, interprets the signals as pain, allowing you to consciously perceive the sensation and respond accordingly.

Wrist to Brain. The following are stimulation points for use with the apparatus and methods of the present disclosure, including both dynamic and static stimulation points as shown in FIGS. 17A and 17B.

Pain Receptors Activation: Pain in the wrist activates nociceptors, which are specialized nerve endings that detect harmful stimuli.

Transmission to Peripheral Nerves: The nociceptors send electrical signals through peripheral nerves. In the wrist, these signals travel through the median nerve and the ulnar nerve, depending on the exact location of the pain.

Spinal Cord Entry: These nerves carry the pain signals up the arm and into the spinal cord through the dorsal root ganglia, located near the spinal cord.

Spinal Cord Processing: Once in the spinal cord, the signals are transmitted to neurons in the dorsal horn. Here, the signals are processed and can be modulated by other neurons, which can either amplify or dampen the pain sensation.

Ascending Pathways: The pain signals are then sent up the spinal cord through ascending pathways, primarily the spinothalamic tract. This tract carries the signals to the brainstem and then to the thalamus, which acts as a relay station.

Thalamus Relay: The thalamus processes the pain signals and forwards them to various parts of the brain, including the somatosensory cortex, which is responsible for perceiving and interpreting the pain.

Perception in the Brain: Finally, the somatosensory cortex, located in the parietal lobe of the brain, interprets the signals as pain, allowing you to consciously perceive the sensation and respond accordingly.

Elbow to Brain. The following are stimulation points for use with the apparatus and methods of the present disclosure, including both dynamic and static stimulation points as shown in FIGS. 17A and 17B.

Pain Receptors Activation: Pain in the elbow activates nociceptors, which are specialized nerve endings that detect harmful stimuli.

Transmission to Peripheral Nerves: The nociceptors send electrical signals through peripheral nerves. In the elbow, these signals travel through the ulnar nerve, the median nerve, and the radial nerve, depending on the exact location of the pain.

Spinal Cord Entry: These nerves carry the pain signals up the arm and into the spinal cord through the dorsal root ganglia, located near the spinal cord.

Spinal Cord Processing: Once in the spinal cord, the signals are transmitted to neurons in the dorsal horn. Here, the signals are processed and can be modulated by other neurons, which can either amplify or dampen the pain sensation.

Ascending Pathways: The pain signals are then sent up the spinal cord through ascending pathways, primarily the spinothalamic tract. This tract carries the signals to the brainstem and then to the thalamus, which acts as a relay station.

Thalamus Relay: The thalamus processes the pain signals and forwards them to various parts of the brain, including the somatosensory cortex, which is responsible for perceiving and interpreting the pain.

Perception in the Brain: Finally, the somatosensory cortex, located in the parietal lobe of the brain, interprets the signals as pain, allowing you to consciously perceive the sensation and respond accordingly.

Shoulder to Brain. The following are stimulation points for use with the apparatus and methods of the present disclosure, including both dynamic and static stimulation points as shown in FIGS. 17A and 17B.

Pain Receptors Activation: Pain in the shoulder activates nociceptors, which are specialized nerve endings that detect harmful stimuli.

Transmission to Peripheral Nerves: The nociceptors send electrical signals through peripheral nerves. In the shoulder, these signals travel through the brachial plexus, a network of nerves that includes the axillary nerve, the radial nerve, and the suprascapular nerve.

Spinal Cord Entry: These nerves carry the pain signals from the shoulder to the spinal cord through the dorsal root ganglia, located near the spinal cord.

Spinal Cord Processing: Once in the spinal cord, the signals are transmitted to neurons in the dorsal horn. Here, the signals are processed and can be modulated by other neurons, which can either amplify or dampen the pain sensation.

Ascending Pathways: The pain signals are then sent up the spinal cord through ascending pathways, primarily the spinothalamic tract. This tract carries the signals to the brainstem and then to the thalamus, which acts as a relay station.

Thalamus Relay: The thalamus processes the pain signals and forwards them to various parts of the brain, including the somatosensory cortex, which is responsible for perceiving and interpreting the pain.

Perception in the Brain: Finally, the somatosensory cortex, located in the parietal lobe of the brain, interprets the signals as pain, allowing you to consciously perceive the sensation and respond accordingly.

Neck to Brain. The following are stimulation points for use with the apparatus and methods of the present disclosure, including both dynamic and static stimulation points as shown in FIGS. 17A and 17B.

Pain Receptors Activation: Pain in the back of the neck activates nociceptors, which are specialized nerve endings that detect harmful stimuli.

Transmission to Peripheral Nerves: The nociceptors send electrical signals through peripheral nerves. In the neck, these signals travel through the cervical nerves, specifically the upper cervical nerves (C1-C4).

Spinal Cord Entry: These nerves carry the pain signals from the neck to the spinal cord through the dorsal root ganglia, located near the spinal cord.

Spinal Cord Processing: Once in the spinal cord, the signals are transmitted to neurons in the dorsal horn. Here, the signals are processed and can be modulated by other neurons, which can either amplify or dampen the pain sensation.

Ascending Pathways: The pain signals are then sent up the spinal cord through ascending pathways, primarily the spinothalamic tract. This tract carries the signals to the brainstem and then to the thalamus, which acts as a relay station.

Thalamus Relay: The thalamus processes the pain signals and forwards them to various parts of the brain, including the somatosensory cortex, which is responsible for perceiving and interpreting the pain.

Perception in the Brain: Finally, the somatosensory cortex, located in the parietal lobe of the brain, interprets the signals as pain, allowing you to consciously perceive the sensation and respond accordingly.

Jaw to Brain. The following are stimulation points for use with the apparatus and methods of the present disclosure, including both dynamic and static stimulation points as shown in FIGS. 17A and 17B.

Pain Receptors Activation: Pain in the jaw activates nociceptors, which are specialized nerve endings that detect harmful stimuli.

Transmission to Peripheral Nerves: The nociceptors send electrical signals through peripheral nerves. In the jaw, these signals travel through the trigeminal nerve, which is the main sensory nerve of the face.

Trigeminal Nerve Pathway: The trigeminal nerve has three branches: the ophthalmic, maxillary, and mandibular nerves. Pain in the jaw typically involves the mandibular branch.

Spinal Cord Entry: The pain signals from the trigeminal nerve do not enter the spinal cord directly. Instead, they travel to the trigeminal ganglion, located near the brainstem.

Brainstem Processing: From the trigeminal ganglion, the signals are transmitted to the trigeminal nerve nuclei in the brainstem. Here, the signals are processed and can be modulated by other neurons, which can either amplify or dampen the pain sensation.

Ascending Pathways: The pain signals are then sent to the thalamus via the trigeminothalamic tract. The thalamus acts as a relay station.

Thalamus Relay: The thalamus processes the pain signals and forwards them to various parts of the brain, including the somatosensory cortex, which is responsible for perceiving and interpreting the pain.

Perception in the Brain: Finally, the somatosensory cortex, located in the parietal lobe of the brain, interprets the signals as pain, allowing you to consciously perceive the sensation and respond accordingly.

Scalp to Brain. The following are stimulation points for use with the apparatus and methods of the present disclosure, including both dynamic and static stimulation points as shown in FIGS. 17A and 17B.

Pain Receptors Activation: Pain on the scalp activates nociceptors, which are specialized nerve endings that detect harmful stimuli.

Transmission to Peripheral Nerves: The nociceptors send electrical signals through peripheral nerves. In the scalp, these signals travel through the trigeminal nerve and the cervical nerves (specifically the greater occipital nerve).

Trigeminal Nerve Pathway: For the front and top of the scalp, the pain signals travel through the ophthalmic branch of the trigeminal nerve.

Cervical Nerve Pathway: For the back of the scalp, the pain signals travel through the greater occipital nerve, which is part of the cervical nerves.

Spinal Cord Entry: The pain signals from the greater occipital nerve enter the spinal cord through the dorsal root ganglia, while signals from the trigeminal nerve do not enter the spinal cord directly but travel to the trigeminal ganglion near the brainstem.

Brainstem Processing: From the trigeminal ganglion, the signals are transmitted to the trigeminal nerve nuclei in the brainstem. Here, the signals are processed and can be modulated by other neurons, which can either amplify or dampen the pain sensation.

Ascending Pathways: The pain signals are then sent to the thalamus via the trigeminothalamic tract (for the trigeminal nerve) and the spinothalamic tract (for the cervical nerves). The thalamus acts as a relay station.

Thalamus Relay: The thalamus processes the pain signals and forwards them to various parts of the brain, including the somatosensory cortex, which is responsible for perceiving and interpreting the pain.

Perception in the Brain: Finally, the somatosensory cortex, located in the parietal lobe of the brain, interprets the signals as pain, allowing you to consciously perceive the sensation and respond accordingly.

Abdomen to Brain. The following are stimulation points for use with the apparatus and methods of the present disclosure, including both dynamic and static stimulation points as shown in FIGS. 17A and 17B.

Pain Receptors Activation: Pain in the left side of the abdomen activates nociceptors, which are specialized nerve endings that detect harmful stimuli.

Transmission to Peripheral Nerves: The nociceptors send electrical signals through peripheral nerves. In the abdomen, these signals travel through the intercostal nerves and the lumbar nerves, depending on the exact location of the pain.

Spinal Cord Entry: These nerves carry the pain signals to the spinal cord through the dorsal root ganglia, located near the spinal cord.

Spinal Cord Processing: Once in the spinal cord, the signals are transmitted to neurons in the dorsal horn. Here, the signals are processed and can be modulated by other neurons, which can either amplify or dampen the pain sensation.

Ascending Pathways: The pain signals are then sent up the spinal cord through ascending pathways, primarily the spinothalamic tract. This tract carries the signals to the brainstem and then to the thalamus, which acts as a relay station.

Thalamus Relay: The thalamus processes the pain signals and forwards them to various parts of the brain, including the somatosensory cortex, which is responsible for perceiving and interpreting the pain.

Perception in the Brain: Finally, the somatosensory cortex, located in the parietal lobe of the brain, interprets the signals as pain, allowing you to consciously perceive the sensation and respond accordingly.

Lower Back to Brain. The following are stimulation points for use with the apparatus and methods of the present disclosure, including both dynamic and static stimulation points as shown in FIGS. 17A and 17B.

Pain Receptors Activation: Pain in the lower back activates nociceptors, which are specialized nerve endings that detect harmful stimuli.

Transmission to Peripheral Nerves: The nociceptors send electrical signals through peripheral nerves. In the lower back, these signals travel through the lumbar and sacral nerves.

Spinal Cord Entry: These nerves carry the pain signals to the spinal cord through the dorsal root ganglia, located near the spinal cord.

Spinal Cord Processing: Once in the spinal cord, the signals are transmitted to neurons in the dorsal horn. Here, the signals are processed and can be modulated by other neurons, which can either amplify or dampen the pain sensation.

Ascending Pathways: The pain signals are then sent up the spinal cord through ascending pathways, primarily the spinothalamic tract. This tract carries the signals to the brainstem and then to the thalamus, which acts as a relay station.

Thalamus Relay: The thalamus processes the pain signals and forwards them to various parts of the brain, including the somatosensory cortex, which is responsible for perceiving and interpreting the pain.

Perception in the Brain: Finally, the somatosensory cortex, located in the parietal lobe of the brain, interprets the signals as pain, allowing you to consciously perceive the sensation and respond accordingly.

Cheek to Brain. The following are stimulation points for use with the apparatus and methods of the present disclosure, including both dynamic and static stimulation points as shown in FIGS. 17A and 17B.

Pain Receptors Activation: Pain in the cheek activates nociceptors, which are specialized nerve endings that detect harmful stimuli.

Transmission to Peripheral Nerves: The nociceptors send electrical signals through peripheral nerves. In the cheek, these signals travel through the trigeminal nerve, which is the main sensory nerve of the face.

Trigeminal Nerve Pathway: The trigeminal nerve has three branches: the ophthalmic, maxillary, and mandibular nerves. Pain in the cheek typically involves the maxillary branch.

Spinal Cord Entry: The pain signals from the trigeminal nerve do not enter the spinal cord directly. Instead, they travel to the trigeminal ganglion, located near the brainstem.

Brainstem Processing: From the trigeminal ganglion, the signals are transmitted to the trigeminal nerve nuclei in the brainstem. Here, the signals are processed and can be modulated by other neurons, which can either amplify or dampen the pain sensation.

Ascending Pathways: The pain signals are then sent to the thalamus via the trigeminothalamic tract. The thalamus acts as a relay station.

Thalamus Relay: The thalamus processes the pain signals and forwards them to various parts of the brain, including the somatosensory cortex, which is responsible for perceiving and interpreting the pain.

Perception in the Brain: Finally, the somatosensory cortex, located in the parietal lobe of the brain, interprets the signals as pain, allowing you to consciously perceive the sensation and respond accordingly.

Chin to Brain. The following are stimulation points for use with the apparatus and methods of the present disclosure, including both dynamic and static stimulation points as shown in FIGS. 17A and 17B.

Mental Nerve: The mental nerve is a sensory nerve that provides feeling to the chin, lower lip, and part of the gums. It branches off from the inferior alveolar nerve, which is itself a branch of the mandibular nerve (V3).

Inferior Alveolar Nerve: The mental nerve originates from the inferior alveolar nerve, which travels through the mandibular canal in the lower jaw. Mandibular Nerve (V3): The inferior alveolar nerve is a branch of the mandibular nerve, the third division of the trigeminal nerve (cranial nerve V). The mandibular nerve carries both sensory and motor information.

Trigeminal Ganglion: Sensory signals from the mental nerve travel through the inferior alveolar nerve to the trigeminal ganglion, a collection of nerve cell bodies located near the brainstem.

Brainstem: From the trigeminal ganglion, the sensory signals are transmitted to the brainstem, specifically to the pons.

Thalamus: The signals are then relayed to the thalamus, which acts as a relay station for sensory information.

Cerebral Cortex: Finally, the sensory information reaches the somatosensory cortex in the brain, where it is processed and interpreted, allowing you to perceive sensations from your chin.

Forehead to Brain. The following are stimulation points for use with the apparatus and methods of the present disclosure, including both dynamic and static stimulation points as shown in FIGS. 17A and 17B.

Trigeminal Nerve (CN V): The trigeminal nerve is the fifth cranial nerve and is responsible for sensation in the face and motor functions such as biting and chewing. It has three main branches: ophthalmic (V1), maxillary (V2), and mandibular (V3).

Ophthalmic Branch (V1): This branch of the trigeminal nerve is responsible for transmitting sensory information from the forehead, upper eyelid, and nose. It carries sensations like touch, pain, and temperature.

### Pathway to the Brain:

Sensory Receptors: Sensory receptors in the skin of the forehead detect stimuli.

Ophthalmic Nerve: These signals travel through the ophthalmic nerve, which enters the skull through the superior orbital fissure.

Trigeminal Ganglion: The ophthalmic nerve fibers converge at the trigeminal ganglion, a collection of nerve cell bodies located near the brainstem.

Brainstem: From the trigeminal ganglion, the sensory signals are transmitted to the brainstem, specifically to the pons.

Thalamus: The signals are then relayed to the thalamus, which acts as a relay station for sensory information.

Cerebral Cortex: Finally, the sensory information reaches the somatosensory cortex in the brain, where it is processed and interpreted, allowing you to perceive sensations from your forehead. When sensory signals travel from the forehead to the brain, they involve a series of chemical processes at the synapses between neurons:
1. Action Potential Initiation.

Stimulus Detection: Sensory receptors in the skin of the forehead detect a stimulus.

Action Potential: This detection generates an electrical signal called an action potential, which travels along the sensory neurons.
2. Synaptic Transmission.

Arrival at Synapse: When the action potential reaches the end of a neuron (the presynaptic terminal), it triggers the opening of voltage-gated calcium channels.

Calcium Influx: Calcium ions (Ca²⁺) enter the presynaptic terminal, causing synaptic vesicles filled with neurotransmitters to move toward the synaptic cleft.
3. Neurotransmitter Release.

Vesicle Fusion: The synaptic vesicles fuse with the presynaptic membrane, releasing neurotransmitters into the synaptic cleft.

Diffusion: The neurotransmitters diffuse across the synaptic cleft to the postsynaptic neuron.
4. Neurotransmitter Binding.

Receptor Activation: Neurotransmitters bind to specific receptors on the postsynaptic membrane, causing ion channels to open or close.

Postsynaptic Potential: This binding generates a postsynaptic potential, which can be excitatory or inhibitory depending on the type of neurotransmitter and receptor involved.
5. Signal Propagation.

Continuation of Signal: If the postsynaptic potential is strong enough, it generates a new action potential in the postsynaptic neuron.

Relay to Brain: This process repeats at each synapse along the pathway until the signal reaches the brainstem and thalamus, and finally the somatosensory cortex in the brain.
6. Neurotransmitter Clearance.

Reuptake: Neurotransmitters are often taken back into the presynaptic neuron for reuse.

Enzymatic Degradation: Some neurotransmitters are broken down by enzymes in the synaptic cleft.

Diffusion: Excess neurotransmitters may diffuse away from the synaptic cleft[].

Vagus Nerve to Brain. The following are stimulation points for use with the apparatus and methods of the present disclosure, including both dynamic and static stimulation points as shown in FIGS. 17A and 17B.

The vagus nerve, also known as the 10th cranial nerve (CN X), is a critical component of the parasympathetic nervous system. It has a complex pathway that involves both sensory (afferent) and motor (efferent) fibers. Here's a detailed explanation of its pathway to the brain:

Origin in the Brainstem: The vagus nerve originates from the medulla oblongata in the brainstem. It has several nuclei in the medulla, including the dorsal motor nucleus, the nucleus ambiguous, the solitary nucleus, and the spinal trigeminal nucleus.

Exiting the Skull: The vagus nerve exits the skull through the jugular foramen, traveling alongside the glossopharyngeal (CN IX) and accessory (CN XI) nerves.

### Pathway Through the Neck and Thorax:

Neck: In the neck, the vagus nerve descends within the carotid sheath, positioned between the internal jugular vein and the carotid artery.

Thorax: As it enters the thorax, the right and left vagus nerves take different paths. The left vagus nerve travels anterior to the aortic arch, while the right vagus nerve passes behind the primary bronchus.

### Branches and Functions:

Sensory (Afferent) Pathways: Sensory fibers from the vagus nerve carry information from the heart, lungs, and digestive tract to the brain. These signals are relayed to the nucleus of the solitary tract (NTS) via the nodose ganglion. Motor (Efferent) Pathways: Motor fibers from the vagus nerve control muscles in the pharynx, larynx, and soft palate, as well as parasympathetic functions in the heart, lungs, and digestive organs.

### Relay to the Brain:

Nucleus of the Solitary Tract (NTS): Sensory information from the vagus nerve is processed in the NTS, which is in the medulla oblongata.

Higher Brain Centers: From the NTS, signals are transmitted to higher brain centers, including the thalamus and cerebral cortex, where they are further processed and integrated.

The vagus nerve plays a vital role in regulating various bodily functions, including heart rate, digestion, and respiratory rate. Its extensive network ensures that the brain receives continuous updates about the state of the body's internal organs.

Interx Full Body Stimulation Treatment.

Applying a biphasic electrical waveform signal of multiple frequences to multiple low impedance points on the body simultaneously creates a complex interaction of electrical signals traveling through various nerve pathways to the brain. Here's an explanation of how these signals interact and the pathways they take:

Pathways from Each Electrode Location to the Brain. The following are stimulation points for use with the apparatus and methods of the present disclosure, including both dynamic and static stimulation points as shown in FIGS. 17A and 17B.

### Top and Bottom of Foot:

Nerves Involved: The tibial nerve and the sural nerve.

Pathway: Signals travel up the tibial nerve to the sciatic nerve, then to the lumbar plexus, and finally to the spinal cord.

Knee: Nerves Involved: The femoral nerve and the sciatic nerve.

Pathway: Signals travel through the femoral nerve to the lumbar plexus and then to the spinal cord.

Hip: Nerves Involved: The femoral nerve and the sciatic nerve.

Pathway: Signals travel through the lumbar plexus to the spinal cord.

Wrist: Nerves Involved: The median nerve and the ulnar nerve.

Pathway: Signals travel through the brachial plexus to the spinal cord.

Elbow: Nerves Involved: The median nerve, the ulnar nerve, and the radial nerve.

Pathway: Signals travel through the brachial plexus to the spinal cord.

Shoulder: Nerves Involved: The brachial plexus.

Pathway: Signals travel through the brachial plexus to the spinal cord.

### Vagus Nerve on Neck:

Nerves Involved: The vagus nerve.

Pathway: Signals travel directly to the brainstem.

Jaw: Nerves Involved: The mandibular branch of the trigeminal nerve (V3).

Pathway: Signals travel through the trigeminal ganglion to the brainstem.

Scalp: Nerves Involved: The ophthalmic branch of the trigeminal nerve (V1).

Pathway: Signals travel through the trigeminal ganglion to the brainstem.

Abdomen: Nerves Involved: The vagus nerve and the lumbar plexus.

Pathway: Signals travel through the vagus nerve to the brainstem and through the lumbar plexus to the spinal cord.

Lower Back: Nerves Involved: The lumbar plexus and the sacral plexus.

Pathway: Signals travel through the lumbar and sacral plexuses to the spinal cord

Left & Right Cheeks: Nerves Involved: The maxillary branch of the trigeminal nerve (V2).

Pathway: Signals travel through the trigeminal ganglion to the brainstem.

Forehead: Nerves Involved: Trigeminal Nerve (CN V).

Pathway: Ophthalmic Branch (V1): These signals travel through the ophthalmic nerve, which enters the skull through the superior orbital fissure. Trigeminal Ganglion: The ophthalmic nerve fibers converge at the trigeminal ganglion, a collection of nerve cell bodies located near the brainstem. Brainstem: From the trigeminal ganglion, the sensory signals are transmitted to the brainstem, specifically to the pons.

Interaction of Signals. When the biphasic electrical waveform is applied simultaneously to all these locations, the signals will travel through their respective pathways to the spinal cord and brainstem. Here, they will converge and be processed by the central nervous system (CNS). The CNS will integrate these signals, which may result in the following interactions:

Summation: The electrical signals may summate, leading to a stronger overall signal being perceived by the brain.

Interference: Signals from different pathways may interfere with each other, potentially altering the perception of the stimulus.

Modulation: The brain may modulate the signals based on their frequency, amplitude, and the specific pathways they traveled.

Brain Interpretation. The brain interprets these signals based on their origin and the type of sensory information they carry. The somatosensory cortex, located in the parietal lobe, is primarily responsible for processing sensory information from the body. Here's how the brain interprets the signals:

Localization: The brain will determine the location of the stimulus based on the specific nerves and pathways activated.

Intensity: The brain will assess the intensity of the stimulus based on the frequency and amplitude of the electrical signals.

Type of Sensation: The brain will interpret the type of sensation based on the nature of the electrical signals and the receptors activated. When an InterX neurostimulator device applies electrical stimulation to the skin via electrodes, several physiological processes occur in the body. Here's a detailed explanation:
1. Electrical Stimulation and Nerve Activation.

Electrode Placement: Electrodes placed on the skin deliver electrical impulses to underlying nerves.

Action Potentials: These electrical impulses generate action potentials (electrical signals) in the sensory and motor nerves. The frequency and intensity of the stimulation determine the strength and type of response.
2. Signal Transmission.

Peripheral Nerves: The action potentials travel along peripheral nerves towards the spinal cord. For example, if electrodes are placed on the wrist, the signals travel through the median or ulnar nerves.

Spinal Cord: The signals enter the spinal cord through the dorsal roots and ascend towards the brain.
3. Synaptic Transmission.

Neurotransmitter Release: At synapses (junctions between neurons), the action potentials cause the release of neurotransmitters. These chemicals transmit the signal across the synapse to the next neuron.

Signal Propagation: The signal continues to propagate through successive neurons until it reaches the brain.
4. Brain Processing.

Thalamus: The signals are relayed to the thalamus, which acts as a central relay station for sensory information.

Somatosensory Cortex: The thalamus sends the signals to the somatosensory cortex in the parietal lobe of the brain, where they are processed and interpreted as sensations such as touch, pain, or temperature.
5. Physiological Responses.

Pain Modulation: Electrical stimulation can activate inhibitory pathways that reduce the perception of pain.

Autonomic Responses: Stimulation of certain nerves, like the vagus nerve, can influence autonomic functions such as heart rate, digestion, and respiratory rate.
6. Chemical Processes.

Ion Movement: Electrical stimulation causes ions (such as sodium and potassium) to move across nerve cell membranes, generating action potentials.

Neurotransmitter Dynamics: The release and reuptake of neurotransmitters at synapses are crucial for signal transmission and modulation.

The application of electrical stimulation through a neurostimulator device initiates a cascade of electrical and chemical processes that result in the activation of nerves, transmission of signals to the brain, and subsequent interpretation and response by the brain. This can lead to therapeutic effects, including pain relief.

Overall, the simultaneous application of electrical signals to multiple body locations results in a complex integration of sensory information, leading to a unique, intense sensory experience that mitigates pain. This is the basis for the InterX Full Body Stimulation Treatment.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A method of reducing pain or drug dependence from a disease, condition or following a medical procedure of a patient comprises the step of:
providing one or more devices having one or more electrodes connected to one or more electric pulse generators that generates a pulse train and a control for controlling the one or more electric pulse generators;
administering electric pulses with the one or more electrodes to one or more locations on skin of the patient at one or more reflexology zones or to one or more reflexology zones, or a reflexology zone or a homeostatic nerve point upstream, downstream, or opposite a site of pain;
measuring an impedance at the one or more locations; and
administering one or more electric pulses at the one or more locations until a point stimulation is reached.

2. The method of claim 1, further comprising administering the one or more electric pulses with the one or more electrodes to one or more locations on skin of the patient adjacent a prior location of stimulation, and repeating until the point stimulation is reached at a new location; or
determining if two or more treatment location have equal of near equal readings in point stim values, then treat both regions by administering electric pulses to both areas concurrently or in series.

3. The method of claim 1, wherein the method uses 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or 50 devices.

4. The method of claim 1, wherein the electric pulses are provided on one or more subsequent days at the one or more locations with the lower impedance that are adjacent to a source of chronic pain, or the reflexology zone or homeostatic nerve points upstream, downstream, or opposite a site of pain, but not at a site of pain.

5. The method of claim 4, wherein at least one of: the location, a frequency of the electrical pulses, or an energy of the electrical pulses is revised immediately after stimulation, after treating one mor more adjacent areas after treatment, or daily.

6. The method of claim 1, wherein two or more locations for treatment are diagonal, contralateral/opposite side, ipsilateral/same side, or checkerboard; or
the electric pulses are selected based on one or more characteristics of the patients, wherein the characteristics of the patient are selected from age, weight, length of drug use, extent of pain, number of drugs used, surface area of skin with the lower impedance, location of skin with the lower impedance, or length of time that the patient has suffered from the disease or condition.

7. The method of claim 1, wherein the disease or condition is selected from at least one of: musculoskeletal conditions, non-healing conditions, post-surgical conditions, ankylosing spondylitis, chronic pain, back pain, sciatica pain, Ehlers Danlos syndrome, fibromyalgia, rheumatoid arthritis, Bell's Palsy, severe headaches, migraines, neuropathic pain conditions, post-traumatic stress disorder (PTSD), anxiety, depression, systemic inflammation conditions, femoral nerve damage, chronic nerve inflammation of sacrum, chronic nerve inflammation of coccyx, chemotherapy induced neuropathy, phantom limb pain, postural orthostatic tachycardia Syndrome (POTS), complex regional pain syndrome (CRPS)/reflex sympathetic dystrophy (RSD), scoliosis, spondylosis, drug dependency, addiction to pain medication, drug addiction, opioid addiction, insomnia, hypersensitivity, to reduce medication(s), connective tissue diseases, or neuropathy.

8. The method of claim 1, further comprising providing two or more electrodes or sets of electrodes connected to the device, the control having:
a first preset treatment for controlling the electric pulse generator to generate a first set of electric pulses for delivery to the patient through a first set of said electrodes; and
a second preset or multiple presets treatment for controlling the electric pulse generator to generate a second set of electric pulses for delivery to the patient through a second set or multiple of said electrodes; and
applying the electrodes based on at least one of: locations with the lower impedance that are adjacent to a source of chronic pain, or the reflexology zone or homeostatic nerve points upstream, downstream, or opposite a site of pain, but not at the site of pain.

9. The method of claim 1, further comprising at least one of:
directing one or more additional electric pulses to one or more reflexology zones of hands or feet;
mapping the location on a face or spine with highest electrical impedance prior to treatment, and applying the electrodes to one or more head locations, one or more spine locations, one or more scalp locations, or one or more areas of skin with the lowest electrical impedance;
subjecting a location near a source of pain or to a reflexology zone thereof based on the mapping, to electric pulses stimulating an area in the reflexology zone to modulate a response wherein a modulated response is one or more of: reducing addiction, reducing pain, reducing patient anxiety, reducing stress, reducing anxiety, and wherein emitted electric pulses are focused or unfocused electric pulses;
reducing local inflammation by a direct targeting of the one or more electric pulses at or about a site of inflammation;
administering the one or more electric pulses to or in an area of the reflexology zone that modulates a response to the pain to suppress urges to take an addictive medication or opioids, or to reduce withdrawal symptoms;
directing an impedance sensing pulse to a skin of the patient and a control for controlling the pulse generator, the impedance sensing pulse at a level below the level necessary for treatment, the impedance sensing pulse applied to the skin at selected locations to measure the impedance of the body to find active areas;
placing the electrodes on one or more locations with pain or stiffness and at least one of: moving dynamically the electrodes with exercise, or increasing an intensity of stimulation over a location with a level of pain to lower the pain or stiffness; or
moving the one or more electrodes while applying the one or more electric pulses from an initial location of treatment to a position that is at least one of: superior, anterior, posterior, lateral, or inferior from an initial location of treatment.

10. The method of claim 1, wherein emitted electric pulses are spherical, radial, convergent, divergent, planar, near planar, focused or unfocused and the electric pulses are administered invasively or noninvasively.

11. The method of claim 1, wherein one or more repeated treatments occur on a schedule over a period of 3, 4, 5, 6, 7, 10 days, or two or more weeks, and treatments can be repeated over time for drug use reduction, pain prevention, reduction of symptoms, anxiety, insomnia, over longer durations of time between repeated treatments.

12. The method of claim 1, wherein a control that is connected to and controlling an interface allows an operator of the device to select a preset treatment parameter from a menu or menus.

13. The method of claim 1, wherein the one or more devices have two or more outputs that each connected to one or more electrodes or sets of electrodes that might be stationary or dynamic; or the device further comprises a phase detection circuit to measure one or more components of impedance to isolate a capacitance and resistance, and one or more head locations and one or more spine locations that are selected based on the measured impedance.

14. The method of claim 1, wherein a location for treatment is first pulsed at a frequency of 2 to 480 Hz on a first day and delivering at least one preset treatment to the patient, wherein a treatment comprises applying electric pulses to one or more head locations and one or more spine locations at between 2 to 480 Hz on one or more subsequent days, until there is a reduction in a disease, condition, pain, or use of drugs.

15. The method of claim 1, wherein one or more treatments reduce an amount of a prescription drug or drugs used by the patient is reduced by at least 25, 30, 40, 50, 60, 70, 75, 80, 90, 95%, or 100%.

16. The method of claim 1, wherein a prescription drug or drugs are pain medication(s).

17. A method of reducing pain or drug dependence from a disease, condition or following a medical procedure of a patient comprises the step of:
providing one or more devices having one or more electrodes connected to one or more electric pulse generators that generates a pulse train and a control for controlling the one or more electric pulse generators;
administering electric pulses with the one or more electrodes to one or more locations on skin of the patient at one or more reflexology zones or to one or more reflexology zones, or a reflexology zone or a homeostatic nerve point upstream, downstream, or opposite a site of pain;
measuring an impedance at the one or more locations; and
administering electric pulses at the one or more locations with a lower impedance, wherein the disease or condition is selected from at least one of: musculoskeletal conditions, non-healing conditions, post-surgical conditions, ankylosing spondylitis, back pain, sciatica pain, Ehlers Danlos syndrome, fibromyalgia, rheumatoid arthritis, Bell's Palsy, severe headaches, migraines, neuropathic pain conditions, post-traumatic stress disorder (PTSD), anxiety, depression, systemic inflammation conditions, femoral nerve damage, chronic nerve inflammation of sacrum, chronic nerve inflammation of coccyx, chemotherapy induced neuropathy, phantom limb pain, postural orthostatic tachycardia Syndrome (POTS), complex regional pain syndrome (CRPS)/reflex sympathetic dystrophy (RSD), scoliosis, spondylosis, drug dependency, addiction to pain medication, drug addiction, opioid addiction, insomnia, hypersensitivity, to reduce medication(s), connective tissue diseases, or neuropathy.

18. The method of claim 17, wherein the method uses 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or 50 devices.

19. A non-transitory computer readable medium for use with a device in a method of reducing pain or drug dependence from a disease, condition or following a medical procedure of a patient comprises the step of:
providing one or more devices having one or more electrodes connected to one or more electric pulse generators that generates a pulse train and a control for controlling the one or more electric pulse generators;
administering electric pulses with the one or more electrodes to one or more locations on skin of the patient at one or more reflexology zones or to one or more reflexology zones, or a reflexology zone or a homeostatic nerve point upstream, downstream, or opposite a site of pain;
measuring an impedance at the one or more locations; and
administering electric pulses at the one or more locations until a point stimulation is reached.

20. A computer-readable storage medium comprising instructions stored thereon that, in response to execution by a processor, causes an apparatus to deliver one or more pulses in a method of reducing pain or drug dependence from a disease, condition or following a medical procedure of a patient comprises the step of:
providing one or more devices having one or more electrodes connected to one or more electric pulse generators that generates a pulse train and a control for controlling the one or more electric pulse generators;
administering electric pulses with the one or more electrodes to one or more locations on skin of the patient at one or more reflexology zones or to one or more reflexology zones, or a reflexology zone or a homeostatic nerve point upstream, downstream, or opposite a site of pain;
measuring an impedance at the one or more locations; and
administering electric pulses at the one or more locations until a point stimulation is reached.
